(19) United States
(12) Patent Application Publication
(10) Pub. No.: US 2024/0132524 A1
(43) Pub. Date: Apr. 25, 2024

(54) SMALL MOLECULES FOR BORON NEUTRON CAPTURE THERAPY

(71) Applicant: Aviko Radiopharmaceuticals, LLC, New York, NY (US)

(72) Inventors: Andrew William Stamford, Chatham, NJ (US); Zhuming Zhang, Hillsborough, NJ (US)

(21) Appl. No.: 18/370,678

(22) Filed: Sep. 20, 2023

Related U.S. Application Data
(60) Provisional application No. 63/408,561, filed on Sep. 21, 2022.

(51) Int. Cl. C07F 5/02; A61K 41/00; A61K 47/26; A61P 35/00

(52) U.S. Cl. CPC C07F 5/025; A61K 41/0095; A61K 47/26; A61P 35/00

(57) ABSTRACT

Disclosed are compounds, compositions, and methods useful for boron neutron capture therapy.

### RELATED APPLICATION

This application claims benefit of priority to U.S. Provisional Patent Application No. 63/408,561, filed Sep. 21, 2022.

### BACKGROUND

Boron neutron capture therapy (BNCT) is a bimodal cancer therapy that requires a boron-containing molecule that selectively partitions into cancer cells and an external neutron beam directed to the cancer. Capture of thermal neutrons by the ¹⁰B nuclei inside the cancer cells results in nuclear fission to give high-energy alpha particles and recoiling ⁷Li nuclei. The high-energy particles damage the tumor cells resulting in tumor cell death while sparing surrounding healthy tissue. New boron delivery agents that selectively partition into tumor tissue in combination with an external neutron beam directed to the tumor may be useful in boron neutron capture therapy for the treatment of various solid tumors.

4-Borono-L-phenylalanine (BPA) is approved in Japan for use in combination with an external neutron beam device for the treatment of recurrent unresectable head & neck cancer. While BPA has demonstrated therapeutic utility in boron neutron capture therapy, it has limitations. Its selectivity for tumor tissue over healthy tissue and its tumor uptake are considered to meet only the minimal requirements for a successful boron neutron capture therapy agent. Agents that have improved uptake into tumor cells and have an improved tumor:healthy tissue ratio relative to BPA may result in improved anti-cancer efficacy.

### SUMMARY

One aspect of the invention provides compounds, compositions, and methods useful for boron neutron capture therapy.

Accordingly, provided herein is a compound having the structure of Formula (I):

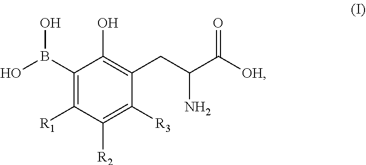

wherein
R₁, R₂, and R₃ are each independently selected from —H, halo, alkyl, haloalkyl, and cycloalkyl; and
the compound is racemic, enriched in one enantiomer, or a single enantiomer;
or a pharmaceutically acceptable salt thereof.

Another aspect of the invention relates to a method of treating cancer, comprising: i) administering to a subject in need thereof a compound of Formula (I) or pharmaceutical composition comprising a compound of Formula (I), wherein the compound accumulates in a plurality of cancer cells in the subject; and ii) irradiating the plurality of cancer cells with neutrons.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon, or of a boron with ¹⁰B-enriched boron, are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl goups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to a cycloalkyl group as hereinbefore defined substituted with at least one halogen.

“Cycloheteroalkyl” refers to a cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl.

“Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. A series of treatments may comprise a second or subsequent treatment weeks to months after a first or preceding treatment.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter ascompared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to a compound of Formula (I):

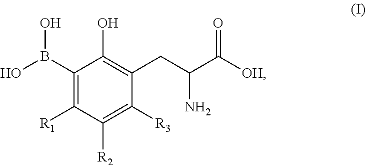

wherein
R₁, R₂, and R₃ are each independently selected from —H, halo, alkyl, haloalkyl, and cycloalkyl; and
the compound is racemic, enriched in one enantiomer, or a single enantiomer;
or a pharmaceutically acceptable salt thereof.

Another aspect of the invention relates to a compound of Formula (IA):

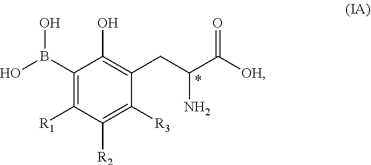

wherein * indicates a chiral carbon with an absolute configuration of (S) or (R); and the compound is not racemic.

In certain embodiments, the absolute configuration of the chiral carbon is (S).

In certain embodiments, the absolute configuration of the chiral carbon is (R).

A further aspect of the invention relates to a compound of Formula (IB):

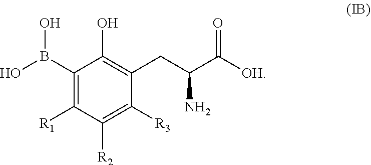

In certain embodiments, a compound having the structure of Formula (IC):

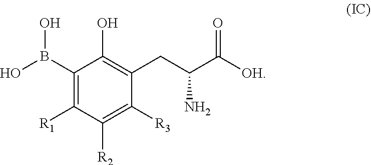

In certain embodiments, R₁, R₂, and R₃ are each independently selected from —H, —Cl, —F, —CF₃, and —CH₃.

In certain embodiments, each of R₁, R₂, and R₃ is not —H.

In certain embodiments, at least one of R₁, R₂, and R₃ is not —H.

In certain embodiments, one and only one of R₁, R₂, and R₃ is not —H.

In certain embodiments, the compound having the structure:

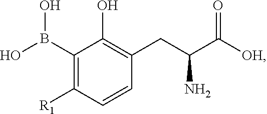

provided R₁ is not —H.

In certain embodiments, the compound having the structure:

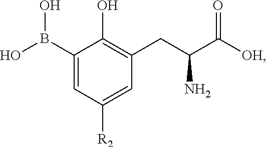

provided R₂ is not —H.

In certain embodiments, the compound having the structure:

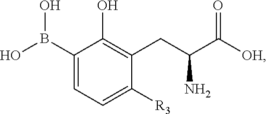

provided R₃ is not —H.

In certain embodiments, the compound having the structure:

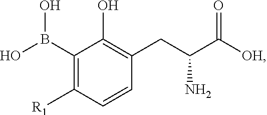

provided R₁ is not —H.

In certain embodiments, the compound having the structure:

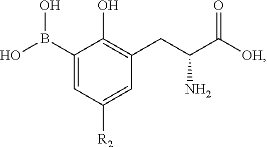

provided R₂ is not —H.

In certain embodiments, the compound having the structure:

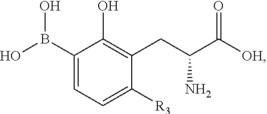

provided R₃ is not —H.

In certain embodiments, the compound having the structure:

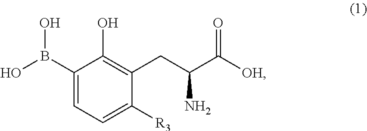

or a pharmaceutically acceptable salt thereof.

In certain embodiments, the compound having the structure selected from:

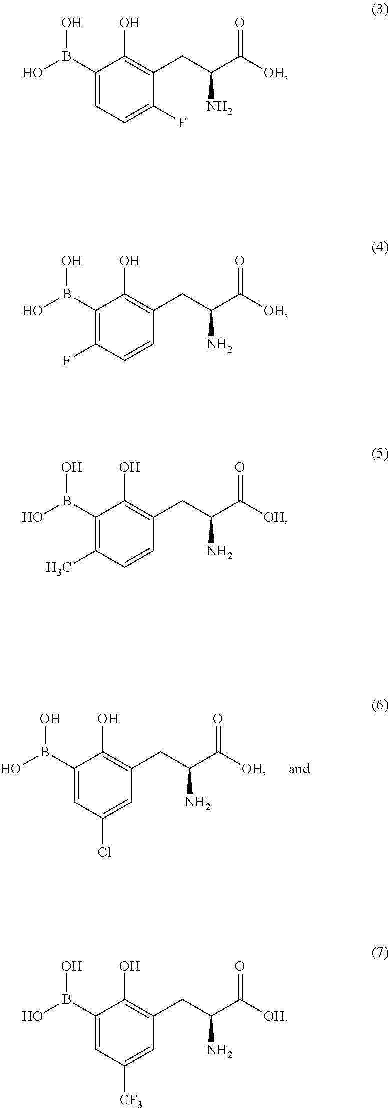 [SMILES: N[C@@H](Cc1c(F)ccc(B(O)O)c1O)C(=O)O ; N[C@@H](Cc1ccc(F)c(B(O)O)c1O)C(=O)O ; Cc1ccc(C[C@H](N)C(=O)O)c(O)c1B(O)O ; N[C@@H](Cc1cc(Cl)cc(B(O)O)c1O)C(=O)O ; "and" ; N[C@@H](Cc1cc(C(F)(F)F)cc(B(O)O)c1O)C(=O)O]

or a pharmaceutically acceptable salt thereof.

In certain embodiments, the compound having the structure selected from:

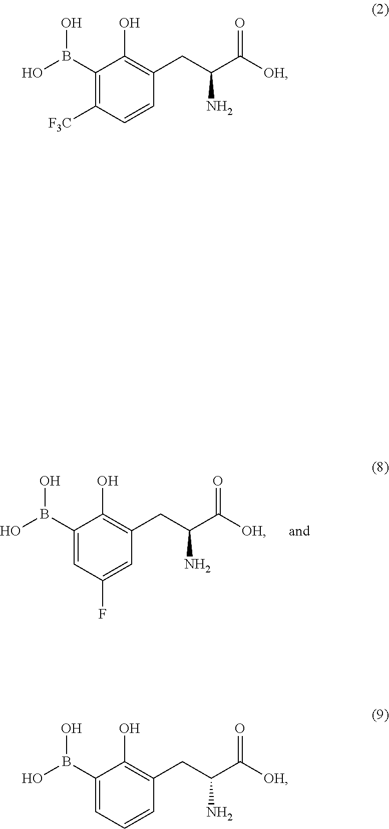 [SMILES: N[C@@H](Cc1ccc(C(F)(F)F)c(B(O)O)c1O)C(=O)O ; N[C@@H](Cc1cc(F)cc(B(O)O)c1O)C(=O)O ; "and" ; N[C@H](Cc1cccc(B(O)O)c1O)C(=O)O]

or a pharmaceutically acceptable salt thereof.

In certain embodiments, the boron atom in the compound is ¹⁰B.

In certain embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. Such compounds are useful, for example, as analytical tools, as probes in biological assays, or as therapeutic agents in accordance with the present invention. For example, in the case of variable R¹, the (C₁-C₄)alkyl or the —O-(C₁-C₄)alkyl can be suitably deuterated (e.g., —CD₃, —OCD₃).

Also within the scope of the invention are compounds produced comprising the natural distribution of ¹¹B and ¹⁰B.

Also within the scope of the invention are compounds enriched in ¹⁰B, i.e., wherein the ¹⁰B is present in abundance of >20%.

Any compound of the invention can also be radiolabed for the preparation of a radiopharmaceutical agent.

### Methods of Treatment

One aspect of the invention provides compounds, compositions, and methods useful for boron neutron capture therapy.

Another aspect of the invention relates to a method of treating cancer, comprising:
i) administering to a subject in need thereof a compound of Formula (I) or a pharmaceutical composition comprising a compound of Formula (I), wherein the compound accumulates in a plurality of cancer cells in the subject; and
ii) irradiating the plurality of cancer cells with neutrons.

Another aspect of the invention relates to a method of treating cancer, comprising:
i) administering to a subject in need thereof a compound of Formula (IA) or a pharmaceutical composition comprising a compound of Formula (IA), wherein the compound accumulates in a plurality of cancer cells in the subject; and
ii) irradiating the plurality of cancer cells with neutrons.

Another aspect of the invention relates to a method of treating cancer, comprising:
i) administering to a subject in need thereof a compound of Formula (IB) or a pharmaceutical composition comprising a compound of Formula (IB), wherein the compound accumulates in a plurality of cancer cells in the subject; and
ii) irradiating the plurality of cancer cells with neutrons.

Another aspect of the invention relates to a method of treating cancer, comprising:
i) administering to a subject in need thereof a compound disclosed herein or a pharmaceutical composition disclosed herein, wherein the compound accumulates in a plurality of cancer cells in the subject; and
ii) irradiating the plurality of cancer cells with neutrons.

In certain embodiments, the compound selectively or preferentially accumulates in the plurality of cancer cells relative to noncancerous cells in the subject.

In certain embodiments, the irradiation results in conversion of a ¹⁰B atom in the compound to an α-particie and a lithium-7 ion.

In certain embodiments, the compound or the composition is administered intravenously.

In certain embodiments, the compound is continually administered during irradiation with neutrons.

In certain embodiments, in step (i) the compound is administered at about 100 mg/kg/h to about 500 mg/kg/h for a first period of time.

In certain embodiments, in step (i) the compound is administered at about 150 mg/kg/h to about 300 mg/kg/h for a first period of time.

In certain embodiments, the first period of time is about 1 hour to about 3 hours. In certain embodiments, the first period of time is about 2 hours.

In certain embodiments, in step (ii) the compound is administered at about 50 mg/kg/h to about 150 mg/kg/h for a second period of time.

In certain embodiments, in step (ii) the compound is administered at about 100 mg/kg/h to about 200 mg/kg/h for a second period of time.

In certain embodiments, the second period of time is about 0.25 hour to about 1.25 hours.

In certain embodiments, the second period of time is about 0.5 to about 1 hours.

In certain embodiments, the cancer is a solid tumor.

In certain embodiments, the cancer is selected from head and neck cancer, glioblastoma, melanoma, sarcoma, breast cancer, meningioma, lung cancer, mesothelioma, hepatocellular carcinoma, and extramammary Paget disease.

In certain embodiments, the cancer is unresectable head and neck cancer.

### Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention.

Pharmaceutical compositions of the invention can be prepared by combining one or more compounds of the invention with a pharmaceutically acceptable carrier and, optionally, one or more additional pharmaceutically active agents.

In certain embodiments, the pharmaceutical composition further comprises a saccharide.

In certain embodiments, the pharmaceutical composition further comprises a polyhydroxy acid.

In certain embodiments, the pharmaceutical composition further comprises a sugar alcohol.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from about 300 mg/kg/day to about 1000 mg/kg/day. In one embodiment, intravenous administration of a compound may typically be from about 400 mg/kg/day to about 600 mg/kg/day. In one embodiment, intravenous administration of a compound may typically be from about 450 mg/kg/day to about 500 mg/kg/day.

Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

Abbreviations used in the instant specification, particularly the schemes and examples, are as follows in Table A:

TABLE A

| Abbreviation | Name |
|---|---|
| ACN or MeCN | Acetonitrile |
| aq. | Aqueous |
| Cbz-Cl | benzyl chloroformate |
| DCM | dichloromethane |
| DMSO | dimethylsulfoxide |
| Dppf or DPPF | 1,1′-Bis(diphenylphosphino)ferrocene |
| EtOAc or EA | ethyl acetate |
| EtOH | ethanol |
| FA | formic acid |
| h or hr(s) | hour or hours |
| HPLC | high performance liquid chromatography |
| MeOH | Methanol |
| MHz | Megahertz |
| min | minute or minutes |
| LCMS | Liquid chromatography mass spectrometry |
| NBS | N-Bromosuccinimide |
| NMR | nuclear magnetic resonance |
| Pd(OH)₂ | palladium hydroxide |
| PE | petrolum ether |
| Pd(dppf)Cl₂ | 1,1′-bis(diphenylphosphino)ferrocene- |
| | palladium(II)dichloride |
| rt or RT | room temperature |
| THF | tetrahydrofuran |

### Example 1. Synthetc Routes to Compounds

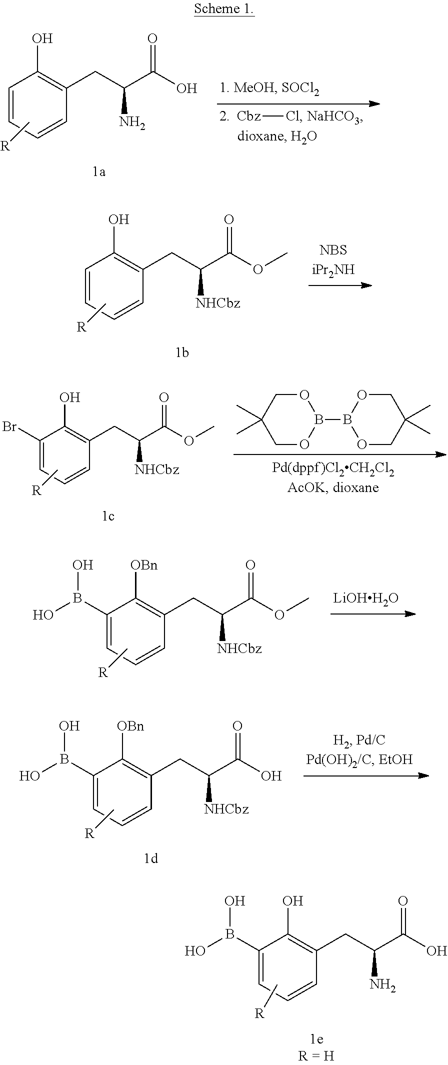

Scheme 1 provides a general synthetic route to the compounds disclosed herein. In Scheme 1, compound 1a is converted to the methyl ester with thionyl chloride in methanol, then the amine of the intermediate methyl ester is protected as a benzyl carbamate by reaction with benzyl chloroformate under basic conditions. The resultant product 1b is brominated with NBS to afford brominated product 1c. Miyaura borylation of 1c followed by aqueous basic hydrolysis affords 1d. Compound 1d is then subjected to hydrogenolysis to afford the product 1e.

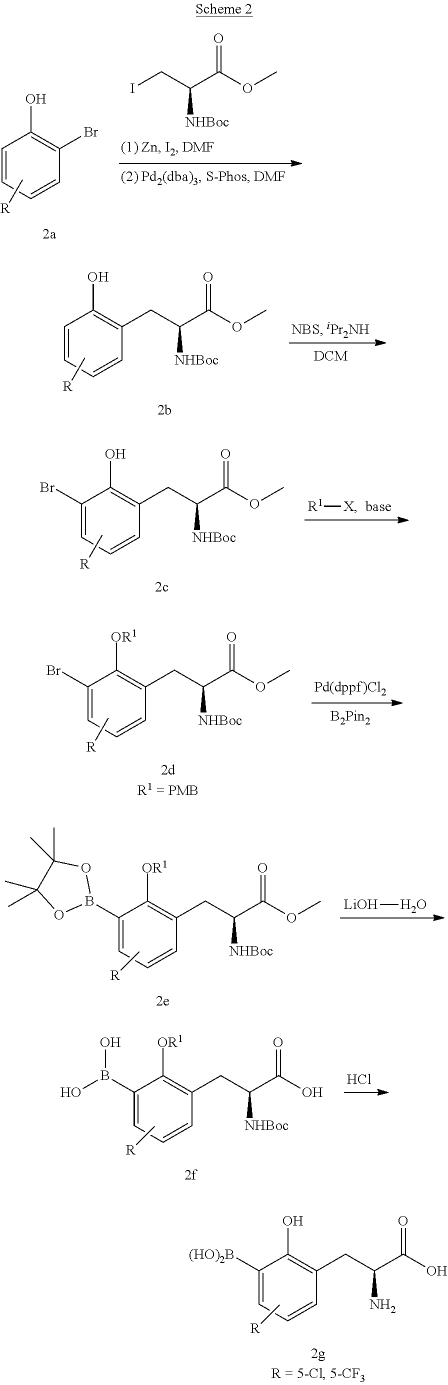

Scheme 2 provides a general synthetic route to the 5-substituted compounds disclosed herein. In Scheme 2, Negishi coupling of bromophenol 2a affords hydroxyphenylalanine derivative 2b. Bromination of 2b with NBS gives intermediate 2c the phenol of which is protected by, for example, a para-methoxybenzyl (PMB) group to give 2d. Miyaura borylation of 2d gives borylated derivative 2e which is then deprotected with aqueous base to give 2f, followed by acidic cleavage of the benzyl ether to give product 2g.

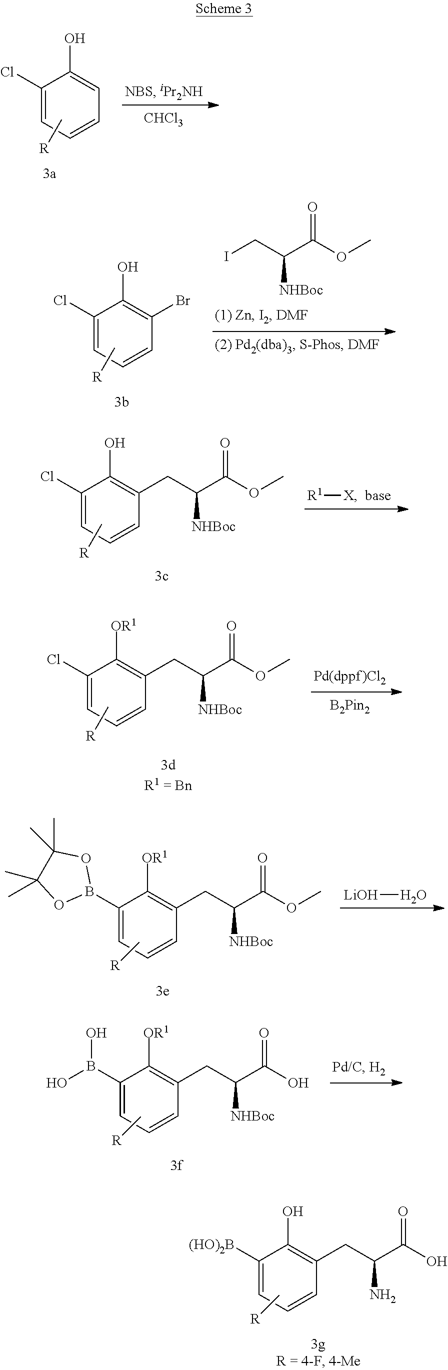

Scheme 3 provides a general synthetic route to the 4-substituted compounds disclosed herein. In Scheme 3, halophenol 3a is brominated with NBS to give 3b. Negishi reaction of 3b affords the protected hydroxyphenylalanine derivative 3c. Protection of the phenol of 3c with, for example, a benzyl group affords benzyl ether 3d. Miyaura borylation of 3d gives the borylated derivative 3e which is then deprotected with aqueous base to give 3f, followed by hydrogenolysis of the benzyl ether to give product 3g.

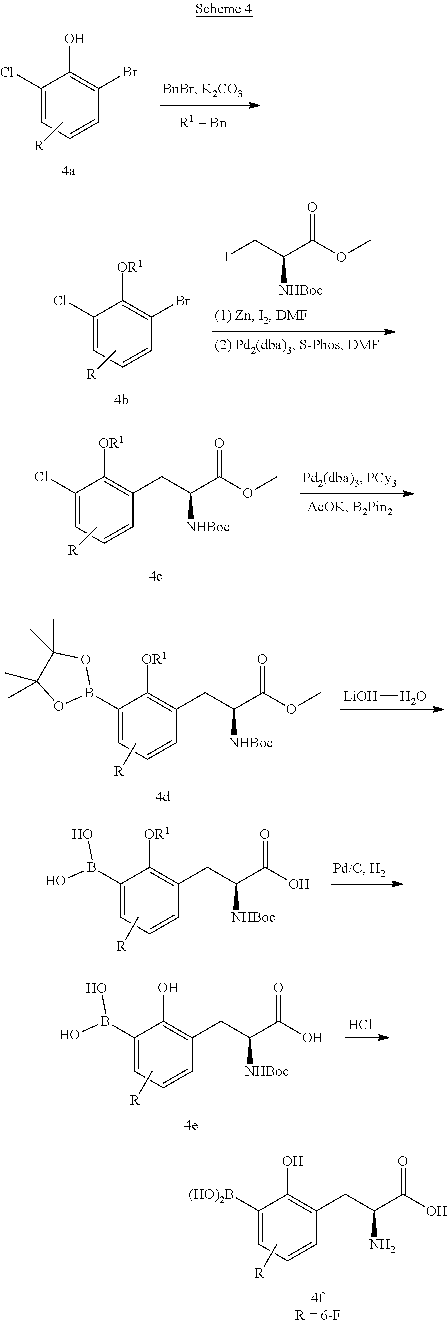

Scheme 4 provides a general synthetic route to the 6-substituted compounds disclosed herein. In Scheme 4, phenol 4a is protected, for example, by benzylation with benzyl bromide to give benzyl ether 4b. Negishi coupling of 4b gives phenylalanine derivative 4c which is then subjected to Miyaura borylation to afford 4d. Basic hydrolysis of 4d followed by hydrogenolysis of the benzyl ether affords intermediate 4e. Treatment of 4e with acid to remove the Boc protecting group gives the product 4f.

### Example 2. Synthesis of Compound 1

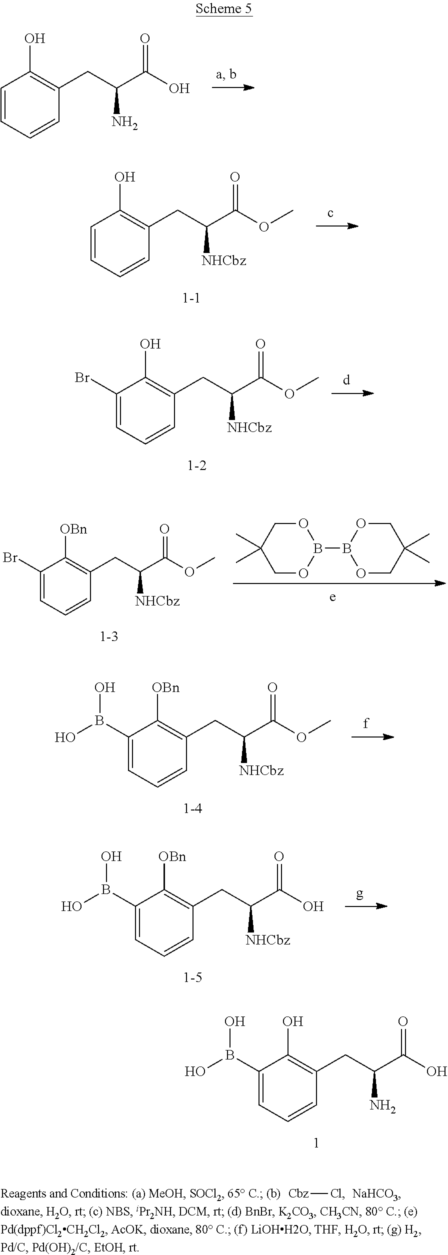

### Preparation of methyl (S)-2-(((benzyloxy)carbonyl)amino)-3-(2-hydroxyphenyl)propanoate (1-1)

Step A: To a stirred solution of 2-hydroxyphenylalanine (2 g, 11.0 mmol, 1.00 equiv) in MeOH (40 mL) at 0° C. was added SOCl₂ (12.0 mL) slowly. The resulting mixture was stirred at 65° C. for 4 h, and then concentrated to afford methyl (25)-2-amino-3-(2-hydroxyphenyl)propanoate as a white solid (2 g, 92.81%).

Step B: To a stirred solution of methyl (25)-2-amino-3-(2-hydroxyphenyl)propanoate (2.5 g, 12.8 mmol, 1.00 equiv) and NaHCO₃ (3.23 g, 38.42 mmol, 3 equiv) in dioxane (15 mL) and H₂O (15 mL) at 0° C. was added Cbz-Cl (2.62 g, 15.37 mmol, 1.2 equiv) dropwise. The resulting mixture was stirred at room temperature for 2 h, and then concentrated. The residue was diluted with water (20 mL) and the mixture was extracted with EA (3×20 mL). The combined organic layers were dried over anhydrous Na₂SO₄, filtered and concentrated. The residue was purified by reversed-phase flash chromatography [with the following conditions: column, C18 silica gel; mobile phase, MeCN in Water (0.1% FA), 50% to 70% gradient in 10 min; detector, UV 220 nm] to afford the title product 1-1 as a yellow solid (2.2 g, 52%).

LCMS (ESI): mass calcd. for C₁₈H₁₉NO₅, 329.1; m/z found, 330.1 [M+H]+.

### Preparation of methyl (S)-2-(((benzyloxy)carbonyl)amino)-3-(3-bromo-2-hydroxyphenyl)propanoate (1-2)

Step C: To a stirred solution of methyl (S)-2-(((benzyloxy)carbonyl)amino)-3-(2-hydroxyphenyl)propanoate (1.5 g, 4.554 mmol, 1 equiv) and diisopropylamine (0.05 g, 0.455 mmol, 0.1 equiv) in DCM (30 mL) at 0° C. was added NBS (0.81 g, 4.554 mmol, 1 equiv) in portions. The resulting mixture was stirred under nitrogen atmosphere at room temperature overnight. Then the mixture was concentrated and the residue was purified by reverse flash chromatography [with the following conditions: column, C18 silica gel; mobile phase, MeCN in Water (0.1% FA), 60% to 80% gradient in 10 min; detector, UV 220 nm] to provide the title product 1-2 as a yellow solid (1.2 g, 65%). LCMS (ESI): mass calcd. for C₁₈H₁₈BrNO₅, 407.0; m/z found, 408 [M+H]⁺.

### Preparation of methyl (S)-3-(2-(benzyloxy)-3-bromophenyl)-2-(((benzyloxy)carbonyl)amino)propanoate (1-3)

Step D: To a stirred solution of methyl (S)-2-(((benzyloxy)carbonyl)amino)-3-(3-bromo-2-hydroxyphenyl)propanoate (900 mg, 2.21 mmol, 1 equiv) and K₂CO₃ (914.0 mg, 6.62 mmol, 3 equiv) in CH₃CN (9 mL) under nitrogen atmosphere was added BnBr (414.8 mg, 2.43 mmol, 1.1 equiv). The resulting mixture was stirred under nitrogen atmosphere at 80° C. for 3 h. Then the mixture was concentrated, diluted with water (20 mL), and extracted with EA (3×15 mL). The combined organic layers were washed with brine (20 mL), dried over anhydrous Na₂SO₄, filtered, and concentrated under vacuum. The residue was purified by reverse flash chromatography [with the following conditions: column, C18 silica gel; mobile phase, MeCN in Water (0.1% FA), 80% to 100% gradient in 10 min; detector, UV 220 nm] to afford the title product 1-3 as a yellow oil (900 mg, 82%). LCMS (ESI): mass calcd. for C₂₅H₂₄BrNO₅, 497.1; m/z found, 498.0 [M+H]+.

### Preparation of (S)-(2-(benzyloxy)-3-(2-(((benzyloxy)carbonyl)amino)-3-methoxy-3-oxopropyl)phenyl)boronic acid (1-4)

Step E: To a stirred mixture of methyl (S)-3-(2-(benzyloxy)-3-bromophenyl)-2-(((benzyloxy)carbonyl)amino)propanoate (900 mg, 1.81 mmol, 1 equiv) and AcOK (531.7 mg, 5.42 mmol, 3 equiv) in 1,4-dioxane (9 mL) were added bis(neopentyl glycolato)diboron (1.22 g, 5.42 mmol, 3 equiv) and Pd(dppf)Cl₂·CH₂Cl₂ (147.11 mg, 0.18 mmol, 0.1 equiv). The resulting mixture was stirred under nitrogen atmosphere at 80° C. overnight. Then the mixture was concentrated, diluted with water (20 mL), and extracted with EA (3×15 mL). The combined organic layers were washed with brine (20 mL), dried over anhydrous Na₂SO₄, filtered, and concentrated. The residue was purified by reverse flash chromatography [with the following conditions: column, C18 silica gel; mobile phase, MeCN in Water (0.1% FA), 60% to 80% gradient in 10 min; detector, UV 220 nm] to provide the title product 1-4 as a yellow solid (600 mg, 72%). LCMS (ESI): mass calcd. for C₂₅H₁₆BNO₇, 463.2 m/z found, 464 [M+H]+.

### Preparation of (S)-3-(2-(benzyloxy)-3-boronophenyl)-2-(((benzyloxy)carbonyl)amino)propanoic acid (1-5)

Step F: To a stirred mixture of (S)-(2-(benzyloxy)-3-(2-(((benzyloxy)carbonyl)amino)-3-methoxy-3-oxopropyl)phenyl)boronic acid (600 mg, 1.30 mmol, 1.00 equiv) in THF (3 mL) and H₂O (3 mL) was added LiOH·H₂O (163.0 mg, 3.89 mmol, 3 equiv). The resulting mixture was stirred at room temperature for 3 h. Then the mixture was acidified to “pH” 5 with 1 M HCl (aq.) and extracted with EA (3×10 mL). The combined organic layers were dried over anhydrous Na₂SO₄, filtered, and concentrated under vacuum. The residue was purified by reverse flash chromatography [with the following conditions: column, C18 silica gel; mobile phase, MeCN in Water (0.1% FA), 60% to 70% gradient in 10 min; detector, UV 254 nm] to afford the title product 1-5 as a yellow oil (500 mg, 86%). LCMS (ESI): mass calcd. for C₂₄H₂₄BNO₇, 449.2 m/z found, 450.2 [M+H]⁺.

### Preparation of (S)-2-amino-3-(3-borono-2-hydroxyphenyl)propanoic acid (1)

Step G: To a solution of (S)-3-(2-(benzyloxy)-3-boronophenyl)-2-(((benzyloxy)carbonyl)amino)propanoic acid (500 mg, 1.11 mmol, 1 equiv) in 40 mL EtOH was added Pd/C (10%, 75 mg) and Pd(OH)₂/C (20%, 75 mg) in a pressure tank. The mixture was hydrogenated under 5 atm of hydrogen pressure at room temperature overnight. Then the mixture was filtered through a Celite pad, and the filtrate was concentrated under reduced pressure. The residue was purified by Prep-HPLC [with the following conditions (Column: Atlantis Prep T3 OBD Column, 19*150 mm 5 μm; Mobile Phase A: Water(0.1% FA), Mobile Phase B: ACN; Flow rate: 25 mL/min; Gradient: to 12% B in 8 min, 12% B; Wave Length: 254/220 nm; RT1(min): 3.9] to afford the title product 1 as an off-white solid (49 mg, 20%). LCMS (ESI): mass calcd. for C₉H₁₂BNO₇, 225.1 m/z found, 226.1 [M+H]⁺; ¹H NMR (300 MHz, D₂O ) δ7.51 (d, J=7.5 Hz, 1H), 7.23 (d, J=7.4 Hz, 1H), 6.87 (t, J=7.5 Hz, 1H), 3.99 (dd, J=7.8, 4.7 Hz, 1H), 3.27 (dd, J=14.4, 4.7 Hz, 1H), 2.96 (dd, J=14.3, 7.8 Hz, 1H) ppm.

### Example 3. Synthesis of Compound 2

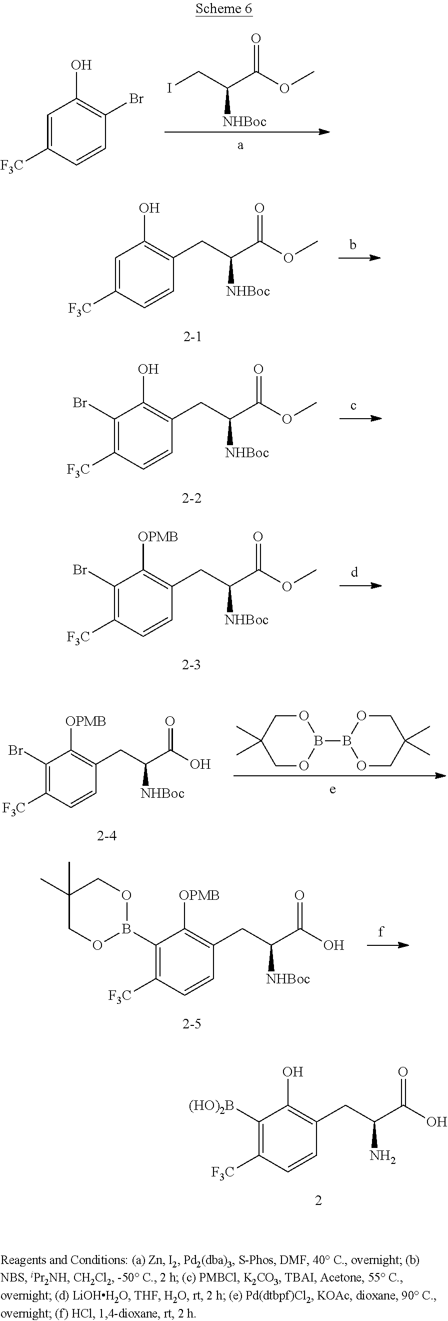

### Preparation of methyl (2S)-2-[(tert-butoxycarbonyl)amino]-3-[2-hydroxy-4-(trifluoromethyl)phenyl]propanoate (2-1)

A mixture of Zn (3.26 g, 49.79 mmol, 4 equiv) and I₂ (0.32 g, 1.25 mmol, 0.1 equiv) in DMF (20 mL) was stirred under nitrogen atmosphere at room temperature for 5 min. To the above mixture was added methyl (2R)-2-[(tert-butoxycarbonyl)amino]-3-iodopropanoate (8.19 g, 24.90 mmol, 2 equiv) in DMF (20 mL) slowly, followed by the addition of I₂ (0.32 g, 1.25 mmol, 0.1 equiv). The resulting mixture was stirred under nitrogen atmosphere at room temperature for 30 min. Then, a suspension of 2-bromo-5-(trifluoromethyl)phenol (3 g, 12.45 mmol, 1 equiv), Pd₂ (dba)₃ (0.85 g, 0.93 mmol, 0.075 equiv), and S-Phos (0.77 g, 1.87 mmol, 0.15 equiv) in DMF (20 mL) was added into the zinc reagent mixture. The resulting mixture was stirred under nitrogen atmosphere at 50° C. for overnight. The reaction was quenched with water (50 mL). The mixture was diluted with EA (100 mL), and filtered through a pad of Celite. The filtrate was washed with brine (3×30 mL), dried over anhydrous Na₂SO₄, filtered, and concentrated. The residue was purified by reversed-phase flash chromatography [with the following conditions: column, C18 silica gel; mobile phase, MeCN in water (0.1% FA), 10% to 70% gradient in 10 min; detector, UV 254 nm] to give the title product as a white solid (2.95 g, 65.2%). LCMS (ESI): mass calcd. for C₁₆H₂₀F₃NO₅, 363.1; m/z found, 362[M−H]⁻.

### Preparation of methyl (2S)-3-[3-bromo-2-hydroxy-4-(trifluoromethyl)phenyl]-2-[(tert-butoxycarbonyl)amino]propanoate (2-2)

To a stirred solution of methyl (2S)-2-[(tert-butoxycarbonyl)amino]-3-[2-hydroxy-4-(trifluoromethyl)phenyl]propanoate (1.7 g, 4.68 mmol, 1 equiv) and bis(propan-2-yl)amine (0.95 g, 9.34 mmol, 2.0 equiv) in DCM (30 mL) at −50° C. was added NBS (0.50 g, 2.81 mmol, 0.60 equiv) in portions. The resulting mixture was stirred under nitrogen atmosphere at −50° C. for 2 h. Then the mixture was filtered, the solid was washed with DCM (2×10 mL). The combined filtrate was concentrated under reduced pressure. The residue was purified by reversed-phase flash chromatography [with the following conditions: column, C18 silica gel; mobile phase, MeCN in water (0.1% FA), 10% to 60% gradient in 10 min; detector, UV 220 nm] to give the title product as a white solid (1.12 g, 54.1%). LCMS (ESI): mass calcd. for C₁₆H₁₉BrF₃NO₅, 441.04; m/z found, 342 [M+H−Boc]⁺.

### Preparation of methyl (2S)-3-{3-bromo-2-[(4-methoxyphenyl)methoxy]-4-(trifluoromethyl) phenyl}-2-[(tert-butoxycarbonyl)amino]propanoate (2-3)

To a stirred mixture of methyl (2S)-3-[3-bromo-2-hydroxy-4-(trifluoromethyl)phenyl]-2-[(tert-butoxycarbonyl)amino]propanoate (1 g, 2.26 mmol, 1 equiv), K₂CO₃ (0.63 g, 4.52 mmol, 2 equiv) and TBAI (0.08 g, 0.23 mmol, 0.1 equiv) in acetone (20 mL) was added 4-methoxybenzyl chloride (0.42 g, 2.71 mmol, 1.20 equiv) slowly. The resulting mixture was stirred under nitrogen atmosphere at 55° C. for overnight. Then the mixture was filtered, the solid was washed with EA (1×10 mL). The combined filtrate was concentrated under reduced pressure. The residue was purified by reversed-phase flash chromatography [with the following conditions: column, C18 silica gel; mobile phase, MeCN in water (0.1% FA), 10% to 90% gradient in 10 min; detector, UV 220 nm] to afford the title product as a light yellow solid (1.18 g, 92.8%). LCMS (ESI): mass calcd. for C₂₄H₂₇BrF₃NO₆, 561.10; m/z found, 584.1[M+23]⁺.

### Preparation of (2S)-3-{3-bromo-2-[(4-methoxyphenyl)methoxy]-4-(trifluoromethyl)phenyl}-2-[(tert-butoxycarbonyl)amino]propanoate (2-4)

To a stirred solution of methyl (2S)-3-{3-bromo-2-[(4-methoxyphenyl)methoxy]-4-(trifluoromethyl)phenyl}-2-[(tert-butoxycarbonyl)amino]propanoate (650 mg, 1.47 mmol, 1 equiv) in THF (8 mL) and H₂O (4 mL) was added LiOH. H₂O (123.4 mg, 2.94 mmol, 2 equiv). The resulting mixture was stirred at room temperature for 2 h. Then the mixture was acidified to “pH 5” with 1 N HCl (aq.), and extracted with EtOAc (3×20 mL). The combined organic layers were washed with brine (1×10 mL), dried over anhydrous Na₂SO₄, filtrated and concentrated under reduced pressure. The residue was purified by reversed-phase flash chromatography [with the following conditions: column, C18 silica gel; mobile phase, MeCN in water (0.1% FA), 10% to 70% gradient in 10 min; detector, UV 220 nm] to afford the title product as a light yellow solid (540 mg, 85.8%). LCMS (ESI): mass calcd. for C₂₃H₂₅BrF₃NO₆, 547.08; m/z found, 546.1[M−H]⁻.

### Preparation of (S)-2-((tert-butoxycarbonyl)amino)-3-(3-(5,5-dimethyl-1,3,2-dioxaborinan-2-yl)-2((4-methoxybenzyl)oxy)-4-(trifluoromethyl)phenyl)propanoic acid (2-5)

To a stirred solution of (2S)-3-{3-bromo-2-[(4-methoxyphenyl)methoxy]-4-(trifluoromethyl)phenyl}-2-[(tert-butoxycarbonyl)amino]propanoic acid (540 mg, 0.99 mmol, 1 equiv) in dioxane (8 mL) were added Pd(dtbpf)Cl₂ (64.2 mg, 0.10 mmol, 0.1 equiv), 2-(5,5-dimethyl-1,3,2-dioxaborinan-2-yl)-5,5-dimethyl-1,3,2-dioxaborinane (444.9 mg, 1.97 mmol, 2 equiv) and KOAc (193.3 mg, 1.97 mmol, 2 equiv). The resulting mixture was stirred under nitrogen atmosphere at 90° C. for overnight. The reaction was quenched with water (20 mL). The mixture was extracted with EtOAc (3×20 mL). The combined organic layers were washed with brine (1×20 mL), dried over anhydrous Na₂SO₄, filtrated and concentrated under reduced pressure. The residue was purified by reversed-phase flash chromatography [with the following conditions: column, C18 silica gel; mobile phase, MeCN in water (0.1% FA), 10% to 70% gradient in 10 min; detector, UV 220 nm] to afford the title product as a light brown oil (201 mg, 35.1%). LCMS (ESI): mass calcd. For C₂₈H₃₅BF₃NO₈, 581.2; m/z found, 482.2[M+H−Boc]⁺.

Preparation of (2S)-2-amino-3-13-(dihydroxyboranyl)-2-hydroxy-4-(trifluoromethyl)phenyllpropanoic acid (2).

To a stirred solution of (S)-2-((tert-butoxycarbonyl)amino)-3-(3-(5,5-dimethyl-1,3,2-dioxaborinan-2-yl)-2((4-methoxybenzyl)oxy)-4-(trifluoromethyl)phenyl)propanoic acid (280 mg, 0.55 mmol, 1 equiv) in dioxane (3 mL) was added HCl in 1,4-dioxane (3 mL, 4 mol/L) dropwise. The resulting mixture was stirred at room temperature for 2 h. Then the mixture was concentrated under reduced pressure. The residue was purified by Prep-TLC [Column: Atlantis Prep T3 OBD Column, 19*150mm 5 μm; Mobile Phase A: water (0.1% FA), Mobile Phase B: ACN; Flow rate: 60 mL/min mL/min; Gradient: isocratic 0% B t13% B in 10 min; Wave Length: 254 nm/220 nm nm; RT1(min): 9.1)] to afford the title product as a white solid (56.2 mg, 35.2%). LCMS (ESI): mass calcd. for C₁₀H₁₁BF₃NO₅, 293.1; m/z found, 294.2[M+H]⁺.

¹H NMR (400 MHz, D₂O) δ7.25 (q, J=8.0 Hz, 2H), 3.98 (dd, J=7.2, 5.4 Hz, 1H), 3.27 (dd, J=14.8, 5.4 Hz, 1H), 3.08 (dd, J=14.7, 7.2 Hz, 1H).

### Example 4. Synthesis of Compound 3

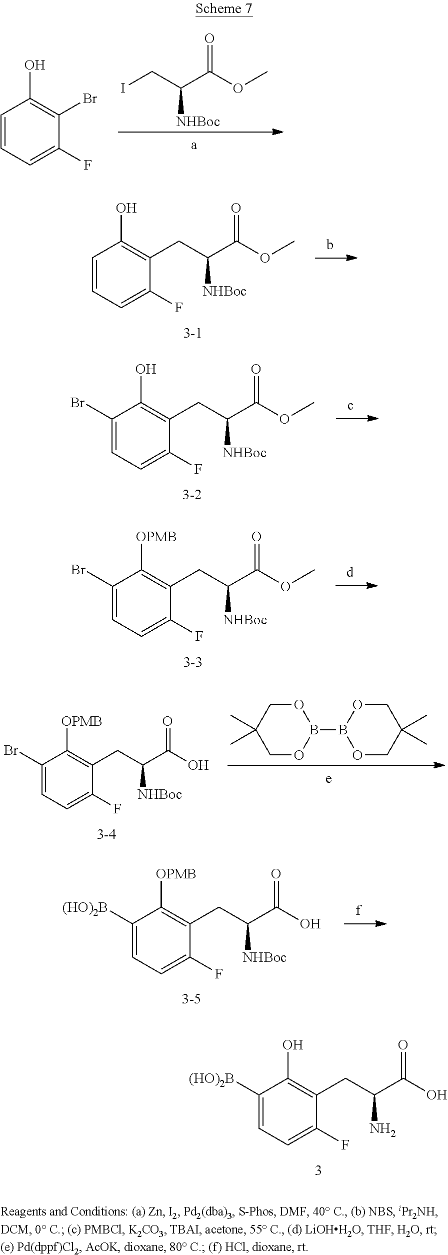

### Preparation of methyl (2S)-2-[(tert-butoxycarbonyl)amino]-3-(2-fluoro-6-hydroxyphenyl) propanoate (3-1)

A mixture of Zn (10.27 g, 157.07 mmol, 3 equiv) and I₂ (1.33 g, 5.23 mmol, 0.1 equiv) in anhydrous DMF (100 mL) was stirred under nitrogen atmosphere at room temperature for 5 min. Then a solution of methyl (2R)-2-[(tert-butoxycarbonyl)amino]-3-iodopropanoate (25.85 g, 78.53 mmol, 1.5 equiv) in DMF (50 mL) was added slowly, followed by the addition of I₂ (1.33 g, 5.23 mmol, 0.1 equiv). The resulting mixture was stirred under nitrogen atmosphere at room temperature for 30 min. A suspension of 2-bromo-3-fluorophenol (10 g, 52.36 mmol, 1 equiv), Pd₂(dba)₃ (3.60 g, 3.93 mmol, 0.075 equiv), and S-Phos (3.22 g, 7.85 mmol, 0.15 equiv) in DMF (50 mL) was added into the zinc reagent mixture. The reaction mixture was stirred under nitrogen atmosphere at 40° C. overnight. After cooling, the reaction was quenched with water (50 mL), diluted with EtOAc (100 mL) and filtered through a pad of Celite. The filtrate was washed with brine (3×50 mL), dried over anhydrous Na₂SO₄, and concentrated. The residue was purified by silica gel column chromatography, eluted with PE/EA (1:1) to afford the title product as a yellow solid (3.9 g, 23.8%). LCMS (ESI): mass calcd. for C₁₅H₂₀FNO₅, 313.13; m/z found, 312.10 [M-1-1] - .

### Preparation of methyl (2S)-3-(3-bromo-6-fluoro-2-hydroxyphenyl)-2-[(tert-butoxycarbonyl) amino]propanoate (3-2)

To a stirred mixture of methyl (2S)-2-[(tert-butoxycarbonyl)amino]-3-(2-fluoro-6-hydroxyphenyl)propanoate (3.9 g, 12.43 mmol, 1 equiv) and bis(propan-2-yl)amine (2.52 g, 24.91 mmol, 2 equiv) in DCM (110 mL) at 0° C. was added NBS (1.33 g, 7.48 mmol, 0.6 equiv). The resulting mixture was stirred under nitrogen atmosphere at 0° C. for 2 h. Then the reaction was quenched with water (60 mL), and extracted with CH₂Cl₂ (3×60 mL). The combined organic layers were washed with brine (1×50 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 10% to 90% gradient in 30 min; detector, UV 220 nm] to give the title product as a yellow solid (2.07 g, 42.4%). LCMS (ESI): mass calcd. for C₁₅H₁₉BrFNO₅, 391.04; m/z found, 390.10 [M−H]⁻.

### Preparation of methyl (2S)-3-{3-bromo-6-fluoro-2-[(4-methoxyphenyl)methoxy]phenyl}-2-[(tert-butoxycarbonyl)amino]propanoate (3-3)

To a stirred mixture of methyl (2S)-3-(3-bromo-6-fluoro-2-hydroxyphenyl)-2-[(tert-butoxycarbonyl)amino]propanoate (2.07 g, 5.38 mmol, 1 equiv), TBAI (195.0 mg, 0.62 mmol, 0.1 equiv) and K₂CO₃ (1.46 g, 10.56 mmol, 2 equiv) in acetone (40 mL) was added 4-methoxybenzyl chloride (991.7 mg, 6.33 mmol, 1.2 equiv). The resulting mixture was stirred under nitrogen atmosphere at 55° C. for overnight. Then reaction was quenched with water (20 mL). The mixture was concentrated and extracted with EtOAc (3×20 mL). The combined organic layers were washed with brine (20 mL), dried over anhydrous Na₂SO₄, filtered, and concentrated under reduced pressure. The residue was purified by silica gel column chromatography eluted with PE/EA (4:1) to give the title product as a yellow solid (2.5 g, 92.6%). LCMS (ESI): mass calcd. for C₂₃H₂₇BrFNO₆, 511.10; m/z found, 512.15 [M+H]⁺.

### Preparation of (2S)-3-{3-bromo-6-fluoro-2-[(4-methoxyphenyl)methoxy]phenyl}-2-[(tert-butoxycarbonyl)amino]propanoic acid (3-4)

To a stirred mixture of methyl (2S)-3-{3-bromo-6-fluoro-2-[(4-methoxyphenyl)methoxy]phenyl}-2-[(tert-butoxycarbonyl)amino]propanoate (2.5 g, 4.88 mmol, 1 equiv) in THF/H₂O (50 mL/25 mL) was added LiOH·H₂O (0.35 g, 14.64 mmol, 3 equiv). The resulting mixture was stirred at room temperature for 3 h. Then the mixture was acidified to “pH” 4 with 2 N HCl (aq.), concentrated, and extracted with EA (3×40 mL). The combined organic layers were washed with brine (1×30 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 10% to 80% gradient in 40 min; detector, UV 220 nm] to afford the title product as a light yellow solid (1.73 g, 71.0%). LCMS (ESI): mass calcd. for C₂₂H₂₅BrFNO₆, 497.08; m/z found, 496.20 [M−H]⁻.

### Preparation of (2S)-2-[(tert-butoxycarbonyl)amino]-3-[3-(dihydroxyboranyl)-6-fluoro-2-[(4-methoxyphenyl)methoxy]phenyl]propanoic acid (3-5)

To a stirred mixture of (2S)-3-{3-bromo-6-fluoro-2-[(4-methoxyphenyl)methoxy]phenyl}-2-[(tert-butoxycarbonyl)amino]propanoic acid (1.5 g, 3.01 mmol, 1 equiv) and 2-(5,5-dimethyl-1,3,2-dioxaborinan-2-yl)-5,5-dimethyl-1,3,2-dioxaborinane (1.02 g, 4.52 mmol, 1.5 equiv) in dioxane (30 mL) were added AcOK (0.59 g, 6.02 mmol, 2 equiv) and Pd(dppf)Cl₂ (0.22 g, 0.30 mmol, 0.1 equiv). The resulting mixture was stirred under nitrogen atmosphere at 80° C. for overnight. The reaction was quenched with water (30 mL). The mixture was extracted with EtOAc (3×30 mL). The combined organic layers were washed with brine (1×30 mL), dried over anhydrous Na₂SO₄, filtered, concentrated under reduced pressure. The residue was purified by reversed-phase flash chromatography [with the following conditions: column, C18 silica gel; mobile phase, MeCN in water (0.1% FA), 10% to 50% gradient in 20 min; detector, UV 220 nm] to provide the title product as a light yellow solid (548 mg, 39.3%). LCMS (ESI): mass calcd. for C₂₂H₂₇BFNO₈, 463.2; m/z found, 462.25 [M−H]⁻.

### Preparation of (2S)-2-amino-3-[3-(dihydroxyboranyl)-6-fluoro-2-hydroxyphenyl]propanoic acid (3)

To a stirred solution of (2S)-2-[(tert-butoxycarbonyl)amino]-3- [3-(dihydroxyboranyl)-6-fluoro-2-[(4-methoxyphenyl)methoxy]phenyl]propanoic acid (548 mg, 1.18 mmol, 1 equiv) in dioxane (5 mL) was added HCl (5 mL, 4 mol/L in dioxane) slowly. The resulting mixture was stirred at room temperature for 3 h. Then the mixture was concentrated. The residue was purified by Prep-HPLC [with the following conditions (Column: Atlantis Prep T3 OBD Column, 19*250mm 10 μm; Mobile Phase A: water (0.1% FA), Mobile Phase B: MeOH-HPLC; Flow rate: 60 mL/min mL/min; Gradient: 2% B to 16% B in 8 min; Wave Length: 254 nm/220 nm; RT1(min): 10.48)] to afford the title product as a white solid (68 mg, 23.3%). LCMS (ESI): mass calcd. for C₉H₁₁BFNO₅, 243.07; m/z found, 244.15 [M+H]⁺. ¹H NMR (400 MHz, D₂O) δ7.50 (t, J=7.8 Hz, 1H), 6.66 (t, J=8.9 Hz, 1H), 3.93 (t, J=6.6 Hz, 1H), 3.20 (dd, J=14.5, 5.2 Hz, 1H), 3.10 (dd, J=14.6, 7.8 Hz, 1H).

### Example 5. Synthesis of Compound 4

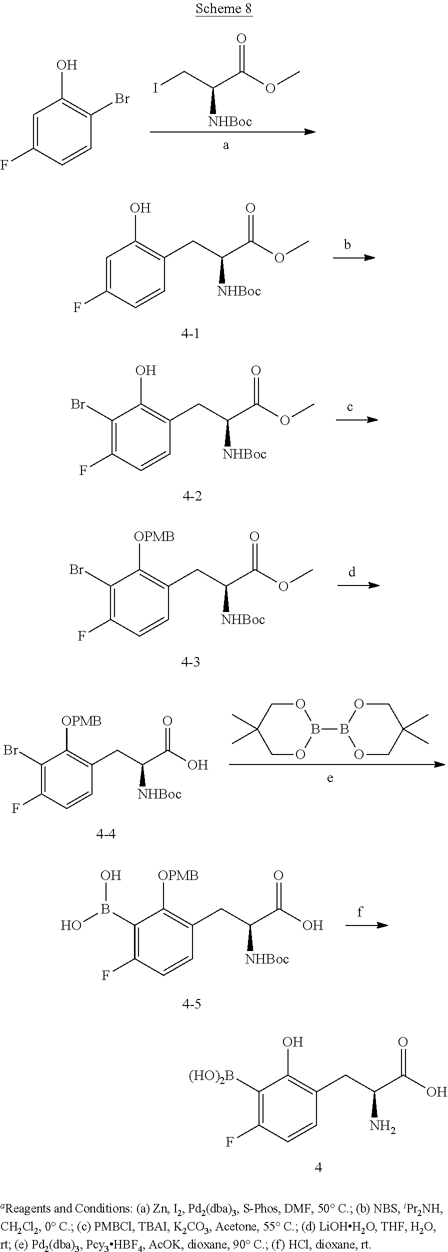

### Preparation of methyl (2S)-2-[(tert-butoxycarbonyl)amino]-3-(4-fluoro-2-hydroxyphenyl)propanoate (4-1)

A mixture of Zn (6.85 g, 104.7 mmol, 4 equiv) and I₂ (1.33 g, 5.2 mmol, 0.2 equiv) in anhydrous DMF (30 mL) was stirred under nitrogen atmosphere at room temperature for 5 min. Then, a solution of methyl (2R)-2-[(tert-butoxycarbonyl)amino]-3-iodopropanoate (10.34 g, 31.4 mmol, 1.2 equiv) in DMF (30 mL) was slowly, followed by the addition of I₂ (1.33 g, 5.2 mmol, 0.2 equiv). The resulting mixture was stirred under nitrogen atmosphere at room temperature for 30 min. A mixture of 2-bromo-5-fluorophenol (5 g, 26.2 mmol, 1 equiv), Pd₂(dba)₃ (2.40 g, 2.6 mmol, 0.1 equiv) and S-Phos (1.61 g, 3.9 mmol, 0.15 equiv) in DMF (30 mL) was added into the zinc reagent mixture. The reaction was stirred under nitrogen atmosphere at 50° C. overnight. After cooling down, the reaction mixture was diluted with water (50 mL), filtered through a Celite pad, and extracted with EtOAc (3×30 mL). The combined organic layers were washed with brine (3×30 mL), dried over anhydrous Na₂SO₄, filtered, and concentrated under reduced pressure. The residue was purified by reversed-phase flash chromatography [with the following conditions: column, C18 silica gel; mobile phase, MeCN in water (10mmol/L NH₄HCO₃), 5% to 50% gradient in 10 min; detector, UV 220 nm] to give the title product as a yellow oil (3.2 g, 39.0%). LCMS (ESI): mass calcd. for C₁₅H₂₀FNO₅, 313.1; m/z found, 312.0 [M−H]⁻.

### Preparation of methyl (2S)-3-(3-bromo-4-fluoro-2-hydroxyphenyl)-2-[(tert-butoxycarbonyl)amino]propanoate (4-2)

To a stirred mixture of methyl (2S)-2-[(tert-butoxycarbonyl)amino]-3-(4-fluoro-2-hydroxyphenyl)propanoate (4.3 g, 13.7 mmol, 1 equiv) and i-Pr₂NH (3.85 mL, 27.45 mmol, 2.00 equiv) in CH₂Cl₂ (40 mL) at 0° C. was added NBS (1.95 g, 10.98 mmol, 0.8 equiv) in portions. The resulting mixture was stirred at 0° C. for 2 h. Then the mixture was concentrated under vacuum and the residue was purified by reversed-phase flash chromatography [with the following conditions: column, C18 silica gel; mobile phase, MeCN in water (0.1% FA), 5% to 55% gradient in 10 min; detector, UV 220 nm] to afford the title product as a yellow solid (3.6 g, 66.9%). LCMS (ESI): mass calcd. for C₁₅H₁₉BrFNO₅, 391.0; m/z found, 389.9 [M−H]⁻.

### Preparation of methyl (2S)-3-{3-bromo-4-fluoro-2-[(4-methoxyphenyl)methoxy]phenyl}-2-[(tert-butoxycarbonyl)amino]propanoate (4-3)

To a stirred mixture of methyl (2S)-3-(3-bromo-4-fluoro-2-hydroxyphenyl)-2-[(tert-butoxycarbonyl)amino]propanoate (3.6 g, 9.18 mmol, 1 equiv), TBAI (0.34 g, 0.92 mmol, 0.1 equiv) and K₂CO₃ (2.54 g, 18.36 mmol, 3 equiv) in acetone (40 mL) was added PMBCl (1.72 g, 11.01 mmol, 1.2 equiv) slowly. The resulting mixture was stirred under nitrogen atmosphere at 55° C. overnight. Then the mixture was concentrated, diluted with water (50 mL) and extracted with EtOAc (3×30 mL). The combined organic layers were washed with brine (1×30 mL), dried over anhydrous Na₂SO₄, filtered, and concentrated under reduced pressure to afford the title product as a brown oil (4.8 g, crude). LCMS (ESI): mass calcd. for C₂₃H₂₇BrFNO₆, 511.1; m/z found, 534.2 [M+Na]⁺.

### Preparation of (2S)-3-{3-bromo-4-fluoro-2-[(4-methoxyphenyl)methoxy]phenyl}-2-[(tert-butoxycarbonyl)amino]propanoic acid (4-4)

To a stirred mixture of methyl (2S)-3-{3-bromo-4-fluoro-2-[(4-methoxyphenyl) methoxy]phenyl}-2-[(tert-butoxycarbonyl)amino]propanoate (4.8 g, crude) in THF (30 mL) and H₂O (15 mL) was added LiOH·H₂O (1.18 g, 28.10 mmol, 3 equiv). The resulting mixture was stirred at room temperature for 2 h. Then the mixture was acidified to “pH” 5 with 1M HCl (aq.), and extracted with EtOAc (3×20 mL). The combined organic layers were washed with brine (1×10 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 5% to 50% gradient in 10 min; detector, UV 220 nm] to afford the title product as a light yellow solid (3.6 g, 77.1%). LCMS (ESI): mass calcd. for C₂₂H₂₅BrFNO₆, 497.1; m/z found, 520.2 [M+Na]⁺

### Preparation of (2S)-2-[(tert-butoxycarbonyl)amino]-3-[3-(dihydroxyboranyl)-4-fluoro-2-[(4-methoxyphenyl)methoxy]phenyl]propanoic acid (4-5)

A mixture of (2S)-3-{3-bromo-4-fluoro-2-[(4-methoxyphenyl)methoxy]phenyl}-2-[(tert-butoxycarbonyl)amino]propanoic acid (2 g, 4.1 mmol, 1 equiv), 2-(5,5-dimethyl-1,3,2-dioxaborinan-2-yl)-5,5-dimethyl-1,3,2-dioxaborinane (1.81 g, 8.1 mmol, 2 equiv), Pd₂(dba)₃ (0.37 g, 0.41 mmol, 0.1 equiv), PCy₃·HBF₄ (0.30 g, 0.81 mmol, 0.2 equiv) and AcOK (1.18 g, 12.1 mmol, 3 equiv) in dioxane (20 mL) was stirred under nitrogen atmosphere at 90° C. overnight. The reaction was quenched with water (30 mL). The mixture was extracted with EtOAc (3×30 mL). The combined organic layers were washed with brine (1×30 mL), dried over anhydrous Na₂SO₄, filtered and concentrated under reduced pressure. The residue was purified by reversed-phase flash chromatography [with the following conditions: column, C18 silica gel; mobile phase, MeCN in water (0.1% FA), 5% to 50% gradient in 10 min; detector, UV 220 nm] to provide the title product as a light yellow solid (1.1 g, 59.2%). LCMS (ESI): mass calcd. for C₂₂H₂₇BFNO₈, 463.2; m/z found, 462.2 [M−H]⁻.

### Preparation of (2S)-2-amino-3-[3-(dihydroxyboranyl)-4-fluoro-2-hydroxyphenyl]propanoic acid (4)

To a stirred solution of (2S)-2-[(tert-butoxycarbonyl)amino]-3-[3-(dihydroxyboranyl)-4-fluoro-2-[(4-methoxyphenyl)methoxy]phenyl]propanoic acid (1.05 g, 2.27 mmol, 1 equiv) in dioxane (5 mL) was added HCl (5 mL, 4 mol/L in 1,4-dioxane). The resulting mixture was stirred at room temperature for 1 h. Then the mixture was concentrated under vacuum. The residue was purified by Prep-HPLC [with the following conditions (Column:)(Bridge Prep Phenyl OBD Column 19*250 mm, 5m; Mobile Phase A: water(0.1% FA), Mobile Phase B: 20 mm NaOH+10% ACN; Flow rate: 60 mL/min mL/min; Gradient: 2% B to 6% B in 10 min; Wave Length: 254 nm/220 nm nm; RT1(min): 6.49)] to afford the title product as a white solid (53.6 mg, 9.4%). LCMS (ESI): mass calcd. for C₉H₁₁BFNO₅, 243.1; m/z found, 244.1 [M+H]⁺; ¹H NMR (400 MHz, D₂O) δ7.19 (t, J=7.6 Hz, 1H), 6.57 (t, J=9.1 Hz, 1H), 4.29 (t, J=6.5 Hz, 1H), 3.27 (dd, J=14.7, 5.4 Hz, 1H), 3.04 (dd, J=14.6, 7.5 Hz, 1H).

### Example 6. Synthesis of Compound 5

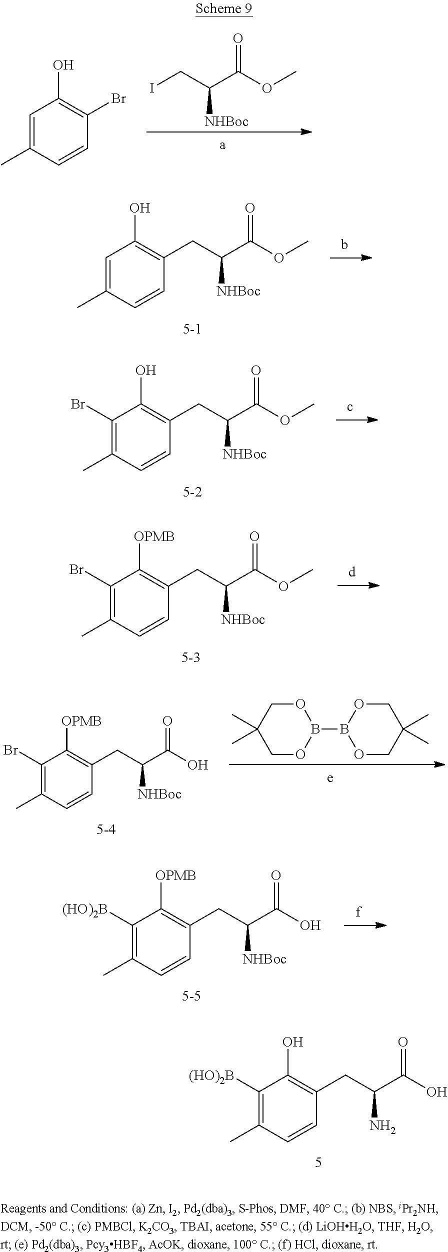

### Preparation of methyl (2S)-2-[(tert-butoxycarbonyl)amino]-3-(2-hydroxy-4-methylphenyl) propanoate (5-1)

A mixture of Zn (4.19 g, 64.16 mmol, 3 equiv) and I₂ (542.8 mg, 2.14 mmol, 0.1 equiv) in anhydrous DMF (40 mL) was stirred under nitrogen atmosphere at room temperature for 5 min. Then a solution of methyl (2R)-2-[(tert-butoxycarbonyl)amino]-3-iodopropanoate (8.45 g, 25.66 mmol, 1.2 equiv) in DMF (40 mL) was added slowly, followed by the addition of I₂ (542.8 mg, 2.14 mmol, 0.1 equiv). The resulting mixture was stirred under nitrogen atmosphere at room temperature for 30 min. A suspension of 2-bromo-5-methylphenol (4 g, 21.39 mmol, 1 equiv), Pd₂(dba)₃ (1.96 g, 2.14 mmol, 0.1 equiv) and S-Phos (1.32 g, 3.21 mmol, 0.15 equiv) in DMF (40 mL) was added into the zinc reagent mixture. The reaction mixture was stirred under nitrogen atmosphere at 40° C. overnight. After cooling, the reaction was quenched with water (50 mL), diluted with EtOAc (100 mL) and filtered through a pad of Celite. The filtrate was washed with brine (3×50 mL), dried over anhydrous Na₂SO₄, and concentrated. The residue was purified by reversed-phase flash chromatography [with the following conditions: column, C18 silica gel; mobile phase, MeCN in water (0.1% FA), 10% to 90% gradient in 30 min; detector, UV 220 nm] to give the title product as a yellow oil (3.2 g, 48.4%). LCMS (ESI): mass calcd. for C₁₆H₂₃NO₅, 309.16; m/z found, 308 [M−H]⁻.

### Preparation of methyl (2S)-3-(3-bromo-2-hydroxy-4-methylphenyl)-2-[(tert-butoxycarbonyl)amino]propanoate (5-2)

To a stirred mixture of methyl (2S)-2-[(tert-butoxycarbonyl)amino]-3-(2-hydroxy-4-methylphenyl)propanoate (3.2 g, 10.34 mmol, 1 equiv) and i-Pr₂NH (2.09 g, 20.69 mmol, 2 equiv) in DCM (60 mL) at −50° C. was added NBS (1.47 g, 8.28 mmol, 0.8 equiv) in portions. The resulting mixture was stirred under nitrogen atmosphere at −50° C. for 2 h. The reaction was quenched with water (60 mL) and extracted with CH₂Cl₂ (3×40 mL). The combined organic layers were washed with brine (40 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 10% to 90% gradient in 30 min; detector, UV 220 nm] to afford the title product as a yellow oil (2.8 g, 69.7%). LCMS (ESI): mass calcd. for C₁₆H₂₂BrNO₅, 387.0; m/z found, 386 [M−H]⁻.

### Preparation of methyl (2S)-3-{3-bromo-2-[(4-methoxyphenyl)methoxy]-4-methylphenyl}-2-[(tert-butoxycarbonyl)amino]propanoate (5-3)

To a stirred mixture of methyl (2S)-3-(3-bromo-2-hydroxy-4-methylphenyl)-2-[(tert-butoxycarbonyl)amino]propanoate (2.8 g, 7.21 mmol, 1 equiv), TBAI (266.4 mg, 0.72 mmol, 0.1 equiv) and K₂CO₃ (1.9 g, 14.42 mmol, 2 equiv) in acetone (45 mL) was added PMBCl (1.35 g, 8.65 mmol, 1.2 equiv). The resulting mixture was stirred under nitrogen atmosphere at 55° C. for overnight. Then reaction was quenched with water (20 mL). The mixture was concentrated, and extracted with EtOAc (3×20 mL). The combined organic layers were washed with brine (20 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 10% to 80% gradient in 40 min; detector, UV 220 nm] to afford the title product as a yellow oil (3.1 g, 84.6%). LCMS (ESI): mass calcd. for C₂₄H₃₀BrNO₆, 507.1; m/z found, 530 [M+Na]⁺.

### Preparation of (2S)-3-{3-bromo-2-[(4-methoxyphenyl)methoxy]-4-methylphenyl}-2-[(tert-butoxycarbonyl)amino]propanoic acid (5-4)

To a stirred mixture of methyl (2S)-3-{3-bromo-2-[(4-methoxyphenyl)methoxy]-4-methylphenyl}-2-[(tert-butoxycarbonyl)amino]propanoate (3.1 g, 6.1 mmol, 1 equiv) in THF/H₂O (20 mL/10 mL) was added LiOH·H₂O (511.7 mg, 12.19 mmol, 2 equiv). The resulting mixture was stirred at room temperature for 3 h. Then the mixture was acidified to “pH” 4 with 2 N HCl (aq.), concentrated, and extracted with EA (3×20 mL). The combined organic layers were washed with brine (1×20 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 10% to 80% gradient in 30 min; detector, UV 220 nm] to afford the title product as a white solid (2.6 g, 86.3%). LCMS (ESI): mass calcd. for C₂₃H₂₈BrNO₆, 493.1; m/z found, 492 [M−H]⁻.

### Preparation of (2S)-2-[(tert-butoxycarbonyl)amino]-3-[3-(dihydroxyboranyl)-2-[(4-methoxyphenyl)methoxy]-4-methylphenyl]propanoic acid (5-5)

To a stirred mixture of (2S)-3-{3-bromo-2-[(4-methoxyphenyl)methoxy]-4-methylphenyl}-2-[(tert-butoxycarbonyl)amino]propanoic acid (2.6 g, 5.26 mmol, 1 equiv) and 2-(5,5-dimethyl-1,3,2-dioxaborinan-2-yl)-5,5-dimethyl-1,3,2-dioxaborinane (2.4 g, 10.52 mmol, 2 equiv) in dioxane (40 mL) were added Pd₂(dba)₃ (481.6 mg, 0.53 mmol, 0.1 equiv), PCy₃·HBF₄ (387.3 mg, 1.05 mmol, 0.2 equiv) and AcOK (1.5 g, 15.78 mmol, 3 equiv). The resulting mixture was stirred under nitrogen atmosphere at 100° C. for overnight. The reaction was quenched with water (30 mL). The mixture was extracted with EtOAc (3×30 mL). The combined organic layers were washed with brine (1×30 mL), dried over anhydrous Na₂SO₄, filtered, concentrated under reduced pressure. The residue was purified by reversed-phase flash chromatography [with the following conditions: column, C18 silica gel; mobile phase, MeCN in water (0.1% FA), 10% to 50% gradient in 20 min; detector, UV 220 nm] to provide the title product as a light yellow solid (1.1 g, 45.5%). LCMS (ESI): mass calcd. for C₂₃H₃₀BNO₈, 459.2; m/z found, 458 [M−H]⁻.

### Preparation of (2S)-2-amino-3-[3-(dihydroxyboranyl)-2-hydroxy-4-methylphenyl]propanoic acid (5)

To a stirred solution of (2S)-2-[(tert-butoxycarbonyl)amino]-3-[3-(dihydroxyboranyl)-2-[(4-methoxyphenyl)methoxy]-4-methylphenyl]propanoic acid (1.1 g, 2.40 mmol, 1 equiv) in dioxane (10 mL) was added HCl (10 mL, 4 mol/L in dioxane) slowly. The resulting mixture was stirred at room temperature for 3 h. Then the mixture was concentrated. The residue was purified by Prep-HPLC [with the following conditions (Column: Atlantis Prep T3 OBD Column, 19*250 mm 10 μm; Mobile Phase A: water(0.1% FA), Mobile Phase B: ACN; Flow rate: 60 mL/min mL/min; Gradient: 0% B to 7% B in 8 min; Wave Length: 254 nm/220 nm nm; RT1(min): 9.47)] to afford the title product as a white solid (34 mg, 5.65%). LCMS (ESI): mass calcd. for C₁₀H₁₄BNO₅, 239.1; m/z found, 240 [M+H]⁺. ¹H NMR (400 MHz, D₂O) δ7.03 (d, J=7.8 Hz, 1H), 6.77 (d, J=7.7 Hz, 1H), 3.98-3.91 (m, 1H), 3.21 (dd, J=14.8, 4.9 Hz, 1H), 2.97 (dd, J=14.7, 7.5 Hz, 1H), 2.24 (s, 3H).

### Example 7. Synthesis of Compound 6

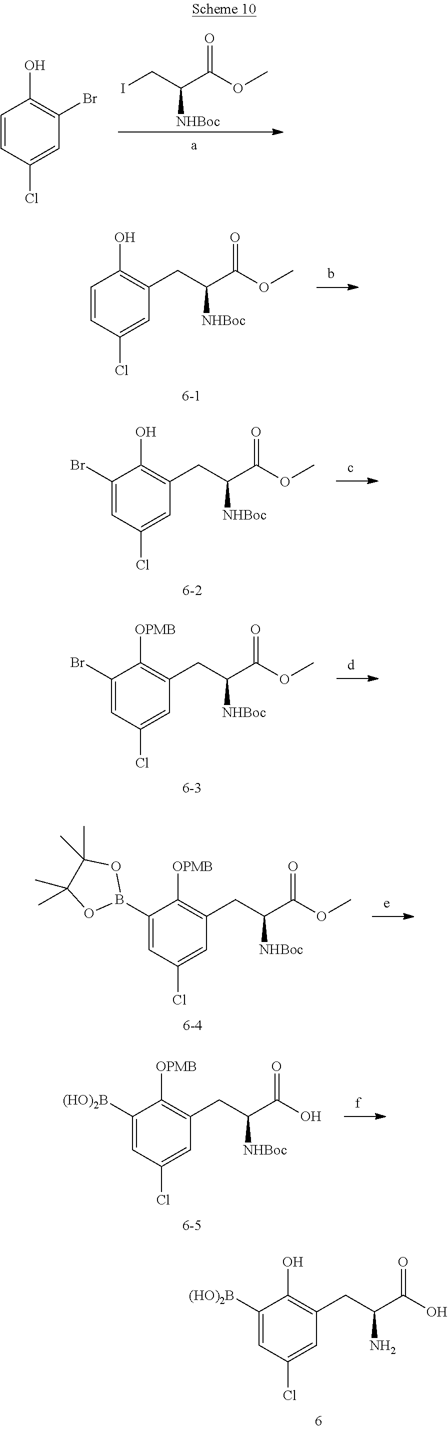

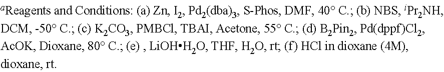

### Preparation of methyl (2S)-2-[(tert-butoxycarbonyl)amino]-3-(5-chloro-2-hydroxyphenyl) propanoate (6-1)

To a stirred mixture of Zn (9.45 g, 144.61 mmol, 3 equiv) in dry DMF (50 mL) was added I₂ (1.22 g, 4.82 mmol, 0.1 equiv). The mixture was stirred under nitrogen atmosphere at room temperature for 5 min. To the above mixture was added a solution of methyl (2R)-2-[(tert-butoxycarbonyl)amino]-3-iodopropanoate (19.04 g, 57.85 mmol, 1.2 equiv) in dry DMF (50 mL) slowly, followed by the addition of I₂ (1.22 g, 4.820 mmol, 0.1 equiv). The resulting mixture was stirred under nitrogen atmosphere at room temperature for additional 0.5 h. Then a suspension of 2-bromo-4-chlorophenol (10 g, 48.20 mmol, 1 equiv), Pd₂(dba)₃ (1.10 g, 1.21 mmol, 0.025 equiv) and S-Phos (0.99 g, 2.41 mmol, 0.05 equiv) in dry DMF (50 mL) was added. The reaction mixture was stirred under nitrogen atmosphere at 40 C overnight. The reaction was quenched by the addition of water (40 mL). The mixture was filtered through a pad of Celite, and the solid was washed with EA (2×50 mL). The filtrate was washed with brine (3×40 mL). The organic layer was dried over anhydrous Na₂SO₄, filtered, concentrated under vacuum. The residue was purified by reverse flash chromatography [with the following conditions: column, C18 silica gel; mobile phase, MeCN in water (0.1% FA), 0% to 100% gradient in 15 min; detector, UV 220 nm] to afford the title product (11 g, 69.2%) as a brown solid. LCMS (ESI): mass calcd. for C₁₅H₂₀ClNO₅, 329.1; m/z found, 230.0 [M−Boc+H]⁺.

### Preparation of methyl (2S)-3-(3-bromo-5-chloro-2-hydroxyphenyl)-2-[(tert-butoxycarbonyl) amino]propanoate (6-2)

To a stirred solution of methyl (2S)-2-[(tert-butoxycarbonyl)amino]-3-(5-chloro-2-hydroxyphenyl)propanoate (5 g, 15.16 mmol, 1 equiv) and ⁱPr₂NH (3.07 g, 30.32 mmol, 2 equiv) in DCM (100 mL) was added NBS (2.16 g, 12.13 mmol, 0.8 equiv) in portions at −50° C. The resulting mixture was stirred under nitrogen atmosphere at −50° C. for 2 h. Then the mixture was diluted with DCM (50 mL), washed with 1 M HCl (aq.) (30 mL) and brine (30 mL). The organic layer was dried over anhydrous Na₂SO₄, filtered, and concentrated under vacuum. The residue was purified by reverse flash chromatography [with the following conditions: column, C18 silica gel; mobile phase, MeCN in water (0.1% FA), 0% to 100% gradient in 15 min; detector, UV 220 nm] to afford the title product (3.8 g, 61.3%) as a yellow oil. LCMS (ESI): mass calcd. for C₁₅H₁₉BrClNO₅, 407.0; m/z found, 308 [M−Boc+H]⁺.

### Preparation of methyl (2S)-3-{3-bromo-5-chloro-2-[(4-methoxyphenyl)methoxy]phenyl}-2-[(tert-butoxycarbonyl)amino]propanoate (6-3)

To a stirred solution of methyl (2S)-3-(3-bromo-5-chloro-2-hydroxyphenyl)-2-[(tert-butoxycarbonyl)amino]propanoate (3.7 g, 9.05 mmol, 1 equiv), K₂CO₃ (2.50 g, 18.11 mmol, 2 equiv) and TBAI (334.4 mg, 0.91 mmol, 0.1 equiv) in acetone (40 mL) was added PMBCl (1.70 g, 10.87 mmol, 1.2 equiv) dropwise. The resulting mixture was stirred at 55° C. overnight. The reaction was quenched by the addition of water (20 mL). The mixture was concentrated and extracted with EA (3×30 mL). The combined organic layers were washed with brine (20 mL), dried over anhydrous Na₂SO₄, filtered, and concentrated under vacuum. The residue was purified by reverse flash chromatography [with the following conditions column, C18 silica gel; mobile phase, MeCN in water (0.1% FA), 0% to 100% gradient in 15 min; detector, UV 220 nm] to afford the title product (3.3 g, 68.9%) as a yellow solid. LCMS (ESI): mass calcd. for C₂₃H₂₇BrClNO₆, 527.0; m/z found, 526.0 [M−H]⁺.

### Preparation of methyl (2S)-2-[(tert-butoxycarbonyl)amino]-3-{5-chloro-2-[(4-methoxyphenyl)methoxy]-3-(4,4,5,5-tetramethyl-1,3,2-dioxaborolan-2-yl)phenyl}propanoate (6-4)

To a stirred solution of methyl (2S)-3-{3-bromo-5-chloro-2-[(4-methoxyphenyl) methoxy]phenyl}-2-[(tert-butoxycarbonyl)amino]propanoate (3.2 g, 6.05 mmol, 1 equiv) in dioxane (50 mL) were added bis(pinacolato)diboron (3.84 g, 15.13 mmol, 2.5 equiv), Pd(dppf)Cl₂ (442.8 mg, 0.61 mmol, 0.1 equiv) and AcOK (1.78 g, 18.15 mmol, 3 equiv) in portions at room temperature. The resulting mixture was stirred under nitrogen atmosphere at 90° C. overnight. The reaction was quenched by the addition of water (20 mL). The mixture was concentrated, and extracted with EA (3×30 mL). The combined organic layers were washed with brine (20 mL), dried over anhydrous Na₂SO₄, filtered, and concentrated under vacuum. The residue was purified by reverse flash chromatography [with the following conditions: column, C18 silica gel; mobile phase, MeCN in water (0.1% FA), 0% to 100% gradient in 15 min; detector, UV 220 nm] to provide the title product (1.7 g, 48.8%) as a yellow solid. LCMS (ESI): mass calcd. for C₂₉H₃₉BClNO₈, 575.2; m/z found, 598.3 [M+Na]⁺.

### Preparation of (2S)-2-[(tert-butoxycarbonyl)amino]-3-[5-chloro-3-(dihydroxyboranyl)-2-[(4-methoxyphenyl)methoxy]phenyl]propanoic acid (6-5)

To a stirred solution of methyl (2S)-2-[(tert-butoxycarbonyl)amino]-3-{5-chloro-2-[(4-methoxyphenyl)methoxy]-3-(4,4,5,5-tetramethyl-1,3,2-dioxaborolan-2-yl)phenyl}propanoate (1.7 g, 2.95 mmol, 1 equiv) in THF (12 mL) and H₂O (6 mL) was added LiOH·H₂O (371.6 mg, 8.86 mmol, 3 equiv) in portions. The resulting mixture was stirred under nitrogen atmosphere at room temperature for 3 h. Then the mixture was acidified to “pH” 5 with 1 N HCl (aq.), and extracted with EA (3×15 mL). The combined organic layers were dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 0% to 100% gradient in 15 min; detector, UV 220 nm] to afford the title product (780 mg, 55.1%) as a white solid. LCMS (ESI): mass calcd. for C₂₂H₂₇BClNO₈, 479.15; m/z found, 478 [M−H]⁺.

### Preparation of ((2S)-2-amino-3-[5-chloro-3-(dihydroxyboranyl)-2-hydroxyphenyl]propanoic acid (6)

To a stirred solution of (2S)-2-[(tert-butoxycarbonyl)amino]-3-[5-chloro-3-(dihydroxyboranyl)-2-[(4-methoxyphenyl)methoxy]phenyl]propanoic acid (700 mg, 1.46 mmol, 1 equiv) in dioxane (6 mL) were added HCl (gas) in 1,4-dioxane (6 mL, 4 mol/L) slowly. The resulting mixture was stirred at room temperature for 2 h. The mixture was concentrated under vacuum and the residue was purified by reverse flash chromatography [with the following conditions: column, C18 silica gel; mobile phase, MeCN in water (0.1% FA), 0% to 100% gradient in 15 min; detector, UV 220 nm] to afford the title product (180 mg, 47.0%) as a white solid. LCMS (ESI): mass calcd. for C₉H₁₁BClNO₅, 259.0; m/z found, 260.1 [M+H]⁺. ¹H NMR (400 MHz, D₂O ) δ7.30 (s, 1H), 7.07 (s, 1H), 4.25-4.14 (m, 1H), 3.19-3.07 (m, 1H), 2.97-2.84 (m, 1H).

### Example 8. Synthesis of Compound 7

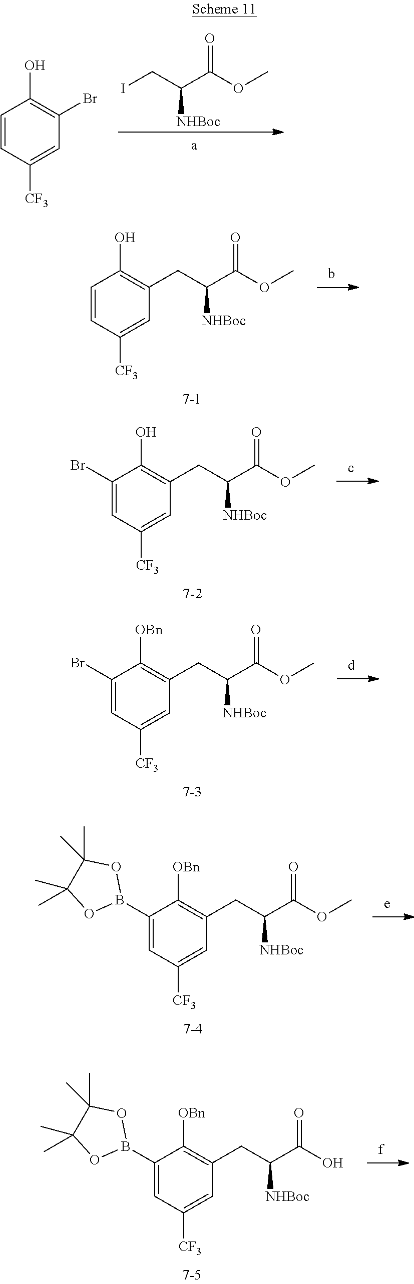

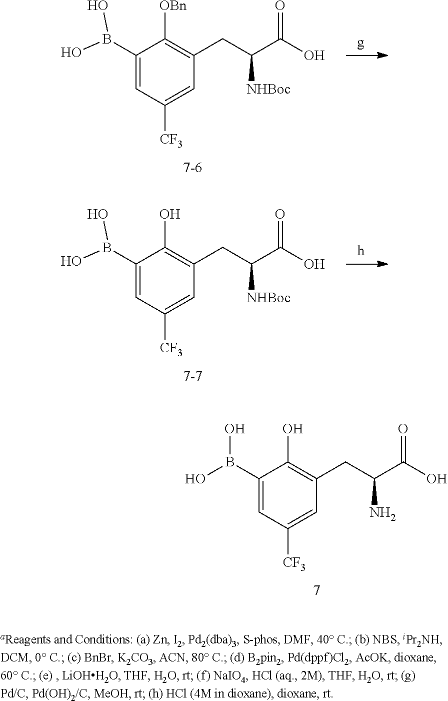 [SMILES: CC(C)(C)OC(=O)N[C@@H](Cc1cc(C(F)(F)F)cc(B(O)O)c1OCc1ccccc1)C(=O)O ; CC(C)(C)OC(=O)N[C@@H](Cc1cc(C(F)(F)F)cc(B(O)O)c1O)C(=O)O ; N[C@@H](Cc1cc(C(F)(F)F)cc(B(O)O)c1O)C(=O)O]

### Preparation of methyl (2S)-2-[(tert-butoxycarbonyl)amino]-3-[2-hydroxy-5-(trifluoromethyl)phenyl]propanoate (7-1)

A mixture of Zn (5.43 g, 82.98 mmol, 4 equiv) and I₂ (1.05 g, 4.15 mmol, 0.2 equiv) in DMF (100 mL) was stirred under nitrogen atmosphere at room temperature for 5 min. To the above mixture was added a solution of methyl (2R)-2-[(tert-butoxycarbonyl)amino]-3-iodopropanoate (8.19 g, 24.90 mmol, 1.2 equiv) in DMF (100 mL) slowly, followed by the addition of I₂ (1.05 g, 4.15 mmol, 0.2 equiv). The resulting mixture was stirred under nitrogen atmosphere at room temperature for additional 30 min. Then, 2-bromo-4-(trifluoromethyl)phenol (5 g, 20.75 mmol, 1 equiv), Pd₂(dba)₃ (1.90 g, 2.08 mmol, 0.1 equiv) and S-Phos (1.28 g, 3.11 mmol, 0.15 equiv) were added into the zinc reagent mixture. The reaction mixture was stirred under nitrogen atmosphere at 40° C. overnight. The reaction was quenched with water (50 mL), diluted with EA (100 mL), and filtered through Celite. The filtrate was washed with brine (3×100 mL), dried over anhydrous Na₂SO₄, filtered, and concentrated. The residue was purified by silica gel column chromatography, eluted with PE/EA (2:1) to give the title product as an off-white solid (4.52 g, 60.0%). LCMS (ESI): mass calcd. for C₁₆H₂₀F₃NO₅, 363.1; m/z found, 264.2 [M+H-Boc]⁺.

### Preparation of methyl (2S)-3-[3-bromo-2-hydroxy-5-(trifluoromethyl)phenyl]-2-[(tert-butoxycarbonyl)amino]propanoate (7-2)

To a stirred solution of methyl (2 S)-2-[(tert-butoxycarbonyl)amino]-3-[2-hydroxy-5-(trifluoromethyl)phenyl]propanoate (4.42 g, 12.17 mmol, 1 equiv) and bis(propan-2-yl)amine (3.41 mL, 24.33 mmol, 2 equiv) in DCM (60 mL) under nitrogen atmosphere at 0° C. was added NBS (2.17 g, 12.17 mmol, 1 equiv) in portions. The resulting mixture was stirred under nitrogen atmosphere at 0° C. for 4 h. Then the mixture was concentrated under reduced pressure and the residue was purified by silica gel column chromatography, eluted with PE/EA (2:1) to give the title product as an off-white solid (3.75 g, 69.7%). LCMS (ESI): mass calcd. for C₁₆H₁₉BrF₃NO₅, 441.0; m/z found, 342.1 [M+H−Boc]⁺

### Preparation of methyl (2S)-3-[2-(benzyloxy)-3-bromo-5-(trifluoromethyl)phenyl]-2-[(tert-butoxycarbonyl)amino]propanoate (7-3)

To a stirred mixture of methyl (2S)-3-[3-bromo-2-hydroxy-5-(trifluoromethyl)phenyl]-2-[(tert-butoxycarbonyl)amino]propanoate (3.65 g, 8.25 mmol, 1 equiv) and K₂CO₃ (3.42 g, 24.76 mmol, 3 equiv) in ACN (70 mL) was added BnBr (1.18 mL, 9.90 mmol, 1.2 equiv). The resulting mixture was stirred at 80° C. overnight. Then the mixture was filtered, the solid was washed with EtOAc (1×10 mL). The filtrate was concentrated under reduced pressure and the residue was purified by silica gel column chromatography, eluted with PE/THF (8:1) to give the title product as a white solid (4.1 g, 96.3%). LCMS (ESI): mass calcd. for C₂₃H₂₅BrF₃NO₅, 531.1; m/z found, 432.1 [M+H−Boc]⁺.

### Preparation of methyl (2S)-3-[2-(benzyloxy)-3-(4,4,5,5-tetramethyl-1,3,2-dioxaborolan-2-yl)-5-(trifluoromethyl)phenyl]-2-[(tert-butoxycarbonyl)amino]propanoate (7-4)

To a stirred mixture of methyl (2S)-3-[2-(benzyloxy)-3-bromo-5-(trifluoromethyl)phenyl]-2-[(tert-butoxycarbonyl)amino]propanoate (4.18 g, 7.85 mmol, 1 equiv) and bis(pinacolato)diboron (4.98 g, 19.63 mmol, 2.5 equiv) in dioxane (80 mL) were added AcOK (2.31 g, 23.56 mmol, 3 equiv) and Pd(dppf)Cl₂ (459.6 mg, 0.63 mmol, 0.08 equiv) at room temperature. The resulting mixture was stirred under nitrogen atmosphere at 60° C. overnight. Then the mixture was filtered, the solid was washed with EtOAc (2×30 mL). The combined filtrate was diluted with water (100 mL) and the mixture was extracted with EtOAc (3×80 mL). The combined organic layers were washed with brine (1×80 mL), dried over anhydrous Na₂SO₄, filtered, and concentrated under reduced pressure. The residue was purified by reversed-phase flash chromatography [with the following conditions: column, C18; mobile phase, MeCN in water (0.1% FA), 50% to 75% gradient in 10 min; detector, UV 220 nm] to afford the title product as a yellow oil (1.97 g, 43.3%). LCMS (ESI): mass calcd. for C₂₉H₃₇BF₃NO₇, 579.3; m/z found, 480.3 [M+H-Boc]⁺.

### Preparation of (2S)-3-12-(benzyloxy)-3-(4,4,5,5-tetramethyl-1,3,2-dioxaborolan-2-yl)-5-(trifluoromethyl)phenyl1-2-1(tert-butoxycarbonyl)aminolpropanoic acid (7-5)

To a stirred mixture of methyl (2S)-3-[2-(benzyloxy)-3-(4,4,5,5-tetramethyl -1,3,2-dioxaborolan-2-yl)-5-(trifluoromethyl)phenyl]-2-[(tert-butoxycarbonyl)amino]propanoate (1.67 g, 2.88 mmol, 1 equiv) in THF (32 mL) /H₂O (16 mL) was added LiOH·H₂O (0.24 g, 5.76 mmol, 2 equiv). The resulting mixture was stirred at room temperature for 3 h. Then the mixture was diluted with water (30 mL), acidified to pH 6 with 2 M HCl (aq.), and extracted with EtOAc (3×50 mL). The combined organic layers were washed with brine (1×50 mL), dried over anhydrous Na₂SO₄, filtered, and concentrated under reduced pressure. This afforded the crude product as a yellow solid (1.71 g, crude). LCMS (ESI): mass calcd. for C₂₈H₃₅BF₃NO₇, 565.3; m/z found, 466.3 [M+H−Boc]⁺.

### Preparation of (2S)-3-[2-(benzyloxy)-3-(dihydroxyboranyl)-5-(trifluoromethyl)phenyl]-2-[(tert-butoxycarbonyl)amino]propanoic acid (7-6)

A mixture of (2 S)-3-[2-(benzyloxy)-3-(4,4,5,5-tetramethyl-1,3,2-dioxaborolan-2-yl)-5-(trifluoromethyl)phenyl]-2-[(tert-butoxycarbonyl)amino]propanoic acid (1.71 g, 3.02 mmol, 1 equiv) and NaIO₄ (1.94 g, 9.07 mmol, 3 equiv) in THF (35 mL)/H₂O (35 mL) was stirred at room temperature for 5 min. Then, HCl (2 M) (1.2 mL) was added to the mixture. The reaction was stirred at room temperature for 2 h. The mixture was filtered, the solid was washed with EtOAc (1×10 mL). The combined filtrate was diluted with water (50 mL), and extracted with EtOAc (3×50 mL). The combined organic layers were washed with brine (1×50 mL), dried over anhydrous Na₂SO₄, filtered, and concentrated under reduced pressure. The residue was purified by reversed-phase flash chromatography [with the following conditions: column, C18; mobile phase, MeCN in water (0.1% FA), 50% to 80% gradient in 10 min; detector, UV 220 nm] to give the title product as an off-white solid (1.21 g, 82.8%). LCMS (ESI): mass calcd. for C₂₂H₂₅BF₃NO₇, 483.2; m/z found, 384.2 [M+H−Boc]⁺.

### Preparation of (2S)-2-[(tert-butoxycarbonyl)amino]-3-[3-(dihydroxyboranyl)-2-hydroxy-5-(trifluoromethyl)phenyl]propanoic acid (7-7)

To a stirred solution of (2S)-3-[2-(benzyloxy)-3-(dihydroxyboranyl)-5-(trifluoromethyl)phenyl]-2-[(tert-butoxycarbonyl)amino]propanoic acid (1.26 g, 2.61 mmol, 1 equiv) in MeOH (100 mL) were added Pd/C (190 mg, 10%) and Pd(OH)₂/C (190 mg, 10%). The resulting mixture was stirred at room temperature overnight under hydrogen atmosphere by using hydrogen balloon. Then the mixture was filtered through a pad of Celite, and the filtrate was concentrated under reduced pressure. The residue was purified by reversed-phase flash chromatography [with the following conditions: column, C18; mobile phase, MeCN in water (0.1% FA), 10% to 60% gradient in 10 min; detector, UV 220 nm] to afford the title product as an off-white solid (631 mg, 61.6%). LCMS (ESI): mass calcd. for C₁₅H₁₉BF₃NO₇, 393.1; m/z found, 294.1 [M+H−Boc]⁺.

### Preparation of (2S)-2-amino-3-[3-(dihydroxyboranyl)-2-hydroxy-5-(trifluoromethyl)phenyl]propanoic acid (7)

To a stirred solution of (2S)-2-[(tert-butoxycarbonyl)amino]-3-[3-(dihydroxyboranyl)-2-hydroxy-5-(trifluoromethyl)phenyl]propanoic acid (300 mg, 0.76 mmol, 1 equiv) in dioxane (6 mL) was added HCl in 1,4-dioxane (6 mL, 4 M) at room temperature. The resulting mixture was stirred at room temperature for 3 h. Then the mixture was concentrated under reduced pressure. The residue was purified by Prep-HPLC [with the following conditions (Column: Xselect CSH F-Phenyl OBD column, 30*250 mm, 5 μm; Mobile Phase A: water(0.1% FA), Mobile Phase B: ACN; Flow rate: 60 mL/min; Gradient: 4% B to 17% B in 10 min; Wave Length: 220/254 nm; RT1(min): 17.05)] to afford the title product as an off-white solid (50.4 mg, 22.1%). LCMS (ESI): mass calcd. for C₁₀H₁₁BF₃NO₅, 293.1; m/z found, 294.1 [M+H]⁺. ¹H NMR (400 MHz, D₂O ) δ7.81 (d, J=2.3 Hz, 1H), 7.52 (d, J=2.4 Hz, 1H), 4.33 (dd, J=7.4, 5.6 Hz, 1H), 3.32 (dd, J=14.5, 5.6 Hz, 1H), 3.13 (dd, J=14.5, 7.4 Hz, 1H).

### Example 9. Synthesis of Compound 8

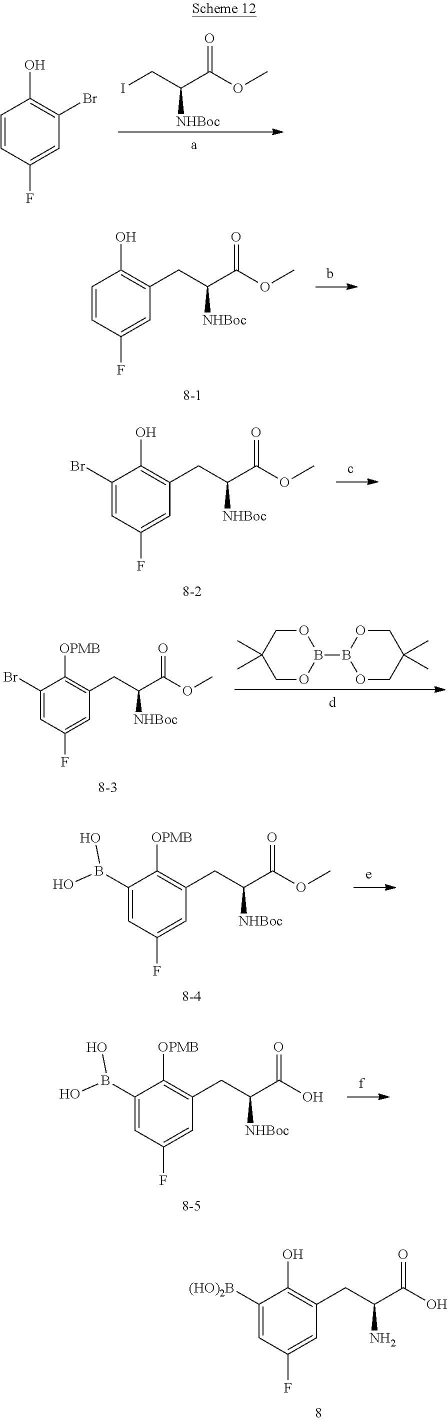

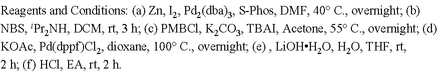

### Preparation of methyl (2S)-2-[(tert-butoxycarbonyl)amino]-3-(5-fluoro-2-hydroxyphenyl)-propanoate (8-1)

A solution of Zn (5.13 g, 78.53 mmol, 3 equiv) and I₂ (0.66 g, 2.62 mmol, 0.1 equiv) in DMF (30 mL) was stirred under nitrogen atmosphere at room temperature for 5 min. Then a solution of methyl (2R)-2-[(tert-butoxycarbonyl)amino]-3-iodopropanoate (10.34 g, 31.414 mmol, 1.2 equiv) in DMF (30 mL) was added slowly, followed by the addition of I₂ (0.66 g, 2.62 mmol, 0.1 equiv). The resulting mixture was stirred under nitrogen atmosphere at room temperature for 30 min. Then, a mixture of 2-bromo-4-fluorophenol (5 g, 26.18 mmol, 1 equiv), Pd₂(dba)₃ (0.60 g, 0.65 mmol, 0.025 equiv) and S-Phos (0.54 g, 1.31 mmol, 0.05 equiv) in DMF (30 mL) was added into the zinc reagent mixture. The reaction mixture was stirred under nitrogen atmosphere at 40° C. overnight. The reaction was quenched with water (50 mL), diluted with EA (50 mL), and filtered through a pad of Celite. The filtrate was washed with brine (3×50 mL), dried over anhydrous Na₂SO₄, filtered, and concentrated. The residue was purified by reversed-phase flash chromatography [with the following conditions: column, C18 silica gel; mobile phase, MeCN in water (0.1% FA), 20% to 40% gradient in 10 min; detector, UV 220 nm] to provide the title product as a yellow oil (3 g, 36.6%). LCMS (ESI): mass calcd. for C₁₅H₂₀FNO₅, 313.13; m/z found, 214.00 [M−Boc+H]⁺.

### Preparation of methyl (2S)-3-(3-bromo-5-fluoro-2-hydroxyphenyl)-2-[(tert-butoxycarbonyl)amino]propanoate (8-2)

To a stirred solution of methyl (2S)-2-[(tert-butoxycarbonyl)amino]-3-(5-fluoro-2-hydroxyphenyl)propanoate (3 g, 9.58 mmol, 1 equiv) and bis(propan-2-yl)amine (0.10 g, 0.958 mmol, 0.1 equiv) in DCM (60 mL) at 0° C. was added NBS (0.85 g, 4.79 mmol, 0.5 equiv) in portions. The resulting mixture was stirred under nitrogen atmosphere at room temperature for 3 h. The reaction was quenched with water (50 mL) and the aqueous layer was extracted with EtOAc (3×50 mL). The combined organic layers were washed with brine (60 mL), dried over anhydrous Na₂SO₄, filtrated, and concentrated under reduced pressure. The residue was purified by reversed-phase flash chromatography [with the following conditions: column, C18 silica gel; mobile phase, MeCN in water (0.1% FA), 40% to 60% gradient in 10 min; detector, UV 220 nm] to provide the title product as a yellow oil (2 g, 53.3%) LCMS (ESI): mass calcd. for C₁₅H₁₉BrFNO₅, 391.04; m/z found, 292 [M−Boc+H]⁺.

### Preparation of methyl (2S)-3-{3-bromo-5-fluoro-2-1(4-methoxyphenyl)methoxylphenyl}-2-[(tert-butoxycarbonyl)amino]propanoate (8-3)

To a stirred mixture of methyl (2S)-3-(3-bromo-5-fluoro-2-hydroxyphenyl)-2-[(tert-butoxycarbonyl)amino]propanoate (2 g, 5.10 mmol, 1 equiv) and K₂CO₃ (1.41 g, 10.20 mmol, 2 equiv) in acetone (40 mL) were added TBAI (0.19 g, 0.51 mmol, 0.1 equiv) and PMBCl (0.96 g, 6.12 mmol, 1.2 equiv) in portions. The resulting mixture was stirred under nitrogen atmosphere at 55° C. for 4 h. Then the mixture was concentrated, diluted with water (50 mL), and extracted with EtOAc (3×50 mL). The combined organic layers were washed with brine (50 mL), dried over anhydrous Na₂SO₄, filtrated, and concentrated under reduced pressure. The residue was purified by reversed-phase flash chromatography [with the following conditions: column, C18 silica gel; mobile phase, MeCN in water (0.1% FA), 50% to 70% gradient in 30 min; detector, UV 254 nm] to provide the title product as a yellow solid (1.53 g, 58.6%). LCMS (ESI): mass calcd. for C₂₃H₂₇BrFNO₆, 511.10; m/z found, 534.20 [M+Na]⁺.

### Preparation of 3-[(2S)-2-[(tert-butoxycarbonyl)amino]-3-methoxy-3-oxopropyl]-5-fluoro-2-[(4-methoxyphenyl)methoxy]phenylboronic acid (8-4)

To a stirred mixture of methyl (2S)-3- {3-bromo-5-fluoro-2-[(4-methoxyphenyl)methoxy]phenyl}-2-[(tert-butoxycarbonyl)amino]propanoate (1.46 g, 2.85 mmol, 1 equiv) and 2-(5,5-dimethyl-1,3,2-dioxaborinan-2-yl)-5,5-dimethyl-1,3,2-dioxaborinane (1.29 g, 5.70 mmol, 2 equiv) in dioxane (30 mL) were added KOAc (839.0 mg, 8.55 mmol, 3 equiv) and Pd(dppf)Cl₂ (208.5 mg, 0.29 mmol, 0.1 equiv) in portions. The resulting mixture was stirred under nitrogen atmosphere at 100° C. overnight. Then the mixture was diluted with water (30 mL), and extracted with EtOAc (3×30 mL). The combined organic layers were washed with brine (30 mL), dried over anhydrous Na2SO4, filtrated, and concentrated under reduced pressure. The residue was purified by silica gel column chromatography, eluted with PE/EA (3:1) to provide the title product as a light brown oil (1.12 g, 82.4%). LCMS (ESI): mass calcd. for C₂₃H₂₉BFNO₈, 477.20; m/z found, 378.25 [M+H−Boc]+.

### Preparation of (2S)-2-[(tert-butoxycarbonyl)amino]-3-[3-(dihydroxyboranyl)-5-fluoro-2-[(4-methoxyphenyl)methoxy]phenyl]propanoic acid (8-5)

A mixture of 3-[(2S)-2-[(tert-butoxycarbonyl)amino]-3-methoxy-3-oxopropyl]-5-fluoro-2-[(4-methoxyphenyl)methoxy]phenylboronic acid (1 g, 2.10 mmol, 1 equiv) and LiOH·H₂O (264.6 mg, 6.30 mmol, 3 equiv) in THF (20 mL) and H₂O (10 mL) was stirred at room temperature for 2 h. Then the mixture was acidified to pH 5 with 1N HCl (aq.), and extracted with EtOAc (3×20 mL). The combined organic layers were washed with brine (20 mL), dried over anhydrous Na₂SO₄, filtered, and concentrated under reduced pressure. The residue was purified by reversed-phase flash chromatography [with the following conditions: column, C18 silica gel; mobile phase, MeCN in water (0.1% FA), 30% to 70% gradient in 20 min; detector, UV 254 nm] to provide the title product as light brown oil (320 mg, 33.0%). LCMS (ESI): mass calcd. for C₂₂H₂₇BFNO₈, 463.18; m/z found, 485.80 [M+Na]⁺.

Preparation of (2S)-2-amino-3-13-(dihydroxyboranyl)-5-fluoro-2-hydroxyphenyllpropanoic acid (8)

To a stirred mixture of (2S)-2-[(tert-butoxycarbonyl)amino]-3-[3-(dihydroxyboranyl)-5-fluoro-2-[(4-methoxyphenyl)methoxy]phenyl]propanoic acid (320 mg, 0.69 mmol, 1 equiv) in EA (1.6 mL) was added HC1 (1.60 mL, 4 mol/L in EA) slowly. The resulting mixture was stirred at room temperature for 2 h. Then the mixture was concentrated under reduced pressure. The residue was purified by reversed-phase flash chromatography [with the following conditions: column, C18 silica gel; mobile phase, MeCN in water (0.1% FA), 0% to 10% gradient in 10 min; detector, UV 254 nm] to provide the title product as an off-white solid (67 mg, 39.9%). LCMS (ESI): mass calcd. for C9H11BFNO5, 243.07; m/z found, 244 [M+H ] +. 1 H NMR (400 MHz, D₂O ) δ6.96 (dd, J =8.9, 3.2 Hz, 1H), 6.77 (dd, J=8.9, 3.2 Hz, 1H), 4.13 (dd, J=7.5, 5.4 Hz, 1H), 3.06 (dd, J=14.6, 5.4 Hz, 1H), 2.84 (dd, J=14.5, 7.5 Hz, 1H).

Example 10. Synthesis of Compound 9

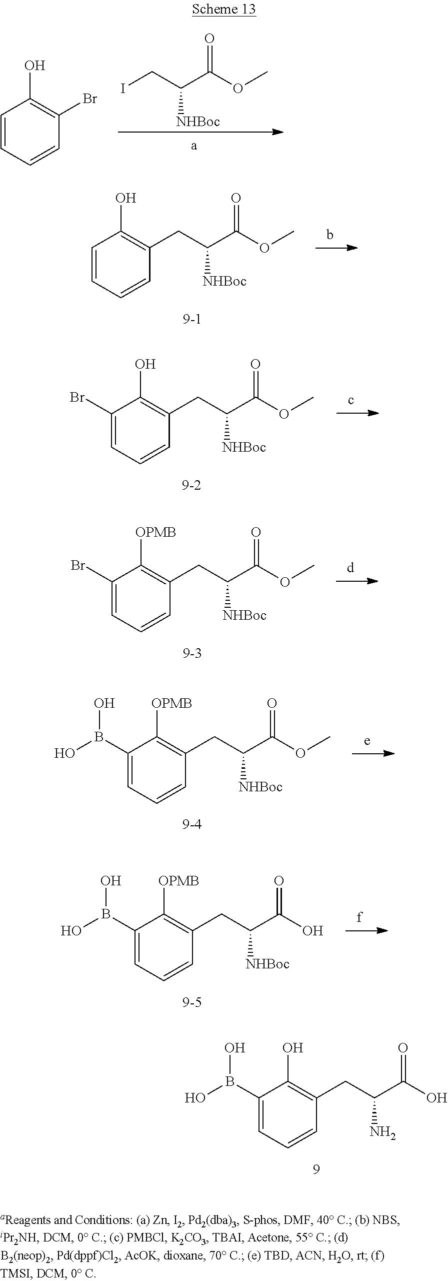

### Preparation of methyl (2R)-2-[(tert-butoxycarbonyl)amino]-3-(2-hydroxyphenyl) propanoate (9-1)

A mixture of Zn (6.05 g, 92.48 mmol, 4 equiv) and I₂ (0.58 g, 2.31 mmol, 0.1 equiv) in DMF (40 mL) was stirred under nitrogen atmosphere at room temperature for 5 min. To the above mixture was added a solution of methyl (2S)-2-[(tert-butoxycarbonyl)amino]-3-iodopropanoate (11.41 g, 34.68 mmol, 1.5 equiv) in DMF (15 mL) slowly, followed by the addition of I₂ (0.58 g, 2.31 mmol, 0.1 equiv). The resulting mixture was stirred under nitrogen atmosphere at room temperature for 30 min. Then to the mixture was added a solution of 2-bromophenol (4 g, 23.12 mmol, 1 equiv) in DMF (10 mL), Pd₂(dba)₃ (2.12 g, 2.31 mmol, 0.1 equiv) and S-phos (1.42 g, 3.47 mmol, 0.15 equiv). The reaction mixture was stirred under nitrogen atmosphere at 40° C. overnight. The reaction was quenched with water (20 mL). The mixture was filtered, the solid was washed with EtOAc (2×10 mL). The filtrate was extracted with EtOAc (3×50 mL). The combined organic layers were washed with water (50 mL) and brine (50 mL), dried over anhydrous Na₂SO₄, filtered, and concentrated under reduced pressure. The residue was purified by reversed-phase flash chromatography [with the following conditions: column, C18; mobile phase, MeCN in water (10mmol/L NH₄HCO₃), 40% to 50% gradient in 5 min; detector, UV 220 nm] to give the title product as a brown yellow oil (4.35 g, 63.7%). LCMS (ESI): mass calcd. for C₁₅H₂₁NO₅, 295.1; m/z found, 196.2 [M+H−Boc]⁺.

### Preparation of methyl (2R)-3-(3-bromo-2-hydroxyphenyl)-2-[(tert-butoxycarbonyl)amino]propanoate (9-2)

To a stirred solution of methyl (2R)-2-[(tert-butoxycarbonyl)amino]-3-(2-hydroxyphenyl)propanoate (4.35 g, 14.73 mmol, 1 equiv) and bis(propan-2-yl)amine (149.0 mg, 1.47 mmol, 0.1 equiv) in DCM (80 mL) under nitrogen atmosphere at 0° C. was added NBS (1.57 g, 8.84 mmol, 0.6 equiv) in portions. The resulting mixture was stirred under nitrogen atmosphere at room temperature overnight. Then the mixture was concentrated under reduced pressure. The residue was purified by reversed-phase flash chromatography [with the following conditions: column, C18; mobile phase, MeCN in water (0.1% FA), 50% to 60% gradient in 6 min; detector, UV 220 nm] to give the title product as a yellow oil (2.75 g, 49.9%). LCMS (ESI): mass calcd. for C₁₅H₂₀BrNO₅, 373.1; m/z found, 274.1 [M+H−Boc]⁺.

### Preparation of methyl (2R)-3-{3-bromo-2-[(4-methoxyphenyl)methoxy]phenyl}-2-[(tert-butoxycarbonyl)amino]propanoate (9-3)

To a stirred mixture of methyl (2R)-3-(3-bromo-2-hydroxyphenyl)-2-[(tert-butoxycarbonyl)amino]propanoate (2.75 g, 7.35 mmol, 1 equiv) and K₂CO₃ (2.03 g, 14.70 mmol, 2 equiv) in acetone (55 mL) were added PMBCl (1.38 g, 8.82 mmol, 1.2 equiv) and TBAI (271.43 mg, 0.74 mmol, 0.1 equiv). The resulting mixture was stirred under nitrogen atmosphere at 55° C. overnight. The reaction was quenched with water at room temperature. The mixture was extracted with EtOAc (3×50 mL). The combined organic layers were washed with brine (1×30 mL), dried over anhydrous Na₂SO₄. After filtration, the filtrate was concentrated under reduced pressure. The residue was purified by reversed-phase flash chromatography [with the following conditions: column, C18; mobile phase, MeCN in water (0.1% FA), 24% to 80% gradient in 10 min; detector, UV 220 nm] to afford the title product as a yellow oil (242 mg, 82.15%). LCMS (ESI): mass calcd. for C₂₃H₂₈BrNO₆, 493.1; m/z found, 516.2 [M+Na]⁺.

### Preparation of 3-[(2R)-2-[(tert-butoxycarbonyl)amino]-3-methoxy-3-oxopropyl]-2-[(4-methoxyphenyl)methoxy]phenylboronic acid (9-4)

To a stirred solution of methyl (2R)-3-[3-bromo-2-[(4-methoxyphenyl)methoxy]phenyl]-2-[(tert-butoxycarbonyl)amino]propanoate (2 g, 4.04 mmol, 1 equiv) and 2-(5,5-dimethyl-1,3,2-dioxaborinan-2-yl)-5,5-dimethyl-1,3,2-dioxaborinane (1.83 g, 8.09 mmol, 2 equiv) in dioxane (40 mL) were added AcOK (1.19 g, 12.13 mmol, 3 equiv) and Pd(dppf)Cl₂ (296.01 mg, 0.41 mmol, 0.1 equiv). The resulting mixture was stirred under nitrogen atmosphere at 70° C. overnight. The mixture was filtered, the filter cake was washed with EtOAc (10 mL). The filtrate was diluted with water (50 mL), and extracted with EtOAc (3×50 mL). The combined organic layers were washed with brine (1×50 mL), dried over anhydrous Na₂SO₄. After filtration, the filtrate was concentrated under reduced pressure. The residue was purified by reversed-phase flash chromatography [with the following conditions: column, C18; mobile phase, MeCN in water (0.1% FA), 50% to 60% gradient in 6 min; detector, UV 220 nm] to give the title product as a yellow oil (1.75 g, 94.18%). LCMS (ESI): mass calcd. for C₂₃H₃₀BNO₈, 459.2; m/z found, 360.2 [M+H−Boc]⁺.

### Preparation of (2R)-2-[(tert-butoxycarbonyl)amino]-3-[3-(dihydroxyboranyl)-2-[(4-methoxyphenyl)methoxy]phenyl]propanoic acid (9-5)

To a stirred mixture of 3-[(2R)-2-[(tert-butoxycarbonyl)amino]-3-methoxy-3-oxopropyl]-2-[(4-methoxyphenyl)methoxy]phenylboronic acid (1.25 g, 2.72 mmol, 1 equiv) in CH₃CN (13 mL) and H₂O (1.3 mL) was added 1H,2H,3H,4H,6H,7H,8H-[1,3]diazino[1,2-a]pyrimidine (757.66 mg, 5.44 mmol, 2 equiv). The resulting mixture was stirred under nitrogen atmosphere at room temperature for 2 h. The mixture was acidified to “pH” 5 with 2N HCl (aq.), and concentrated. The residue was purified by reversed-phase flash chromatography [with the following conditions: column, C18; mobile phase, MeCN in water (0.1% FA), 30% to 50% gradient in 10 min; detector, UV 220 nm] to afford the title product as an off-white solid (389 mg, 32.10%). LCMS (ESI): mass calcd. for C₂₂H₂₈BNO₈, 445.2; m/z found, 444.3 [M−H]⁻.

### Preparation of (2R)-2-amino-3-[3-(dihydroxyboranyl)-2-hydroxyphenyl]propanoic acid; trifluoroacetic acid (9)

To a stirred mixture of 3-[(2R)-2-[(tert-butoxycarbonyl)amino]-3-methoxy-3-oxopropyl]-2-[(4-methoxyphenyl)methoxy]phenylboronic acid (200 mg, 0.44 mmol, 1 equiv) in DCM (5 mL) under nitrogen atmosphere at 0° C. was added TMSI (261.39 mg, 1.31 mmol, 3 equiv) dropwise. The resulting mixture was stirred under nitrogen atmosphere at 0° C. for 2 h. The reaction was quenched by the addition of water (2 mL). The aqueous layer was purified by Prep-HPLC [with the following conditions (Column: Atlantis Prep T3 OBD Column, 19*150mm 5 μm; Mobile Phase A: water (0.05% TFA), Mobile Phase B: ACN; Flow rate: 60 mL/min; Gradient: 0% B to 10% B in 10 min; Wave Length: 254 nm/220 nm; RT1(min): 9.65)] to afford the title product as a white solid (39.0 mg, 25.12%). LCMS (ESI): mass calcd. for C₁₁H₁₃BF₃NO₇, 339.1; m/z found, 226.1 [M+H−TFA]⁺. ¹H NMR (400 MHz, D₂O ) δ7.52 (dd, J=7.5, 1.8 Hz, 1H), 7.24 (dd, J=7.5, 1.8 Hz, 1H), 6.88 (t, J=7.4 Hz, 1H), 4.13-4.01 (m, 1H), 3.29 (dd, J=14.5, 4.8 Hz, 1H), 3.00 (dd, J=14.5, 7.9 Hz, 1H).

### Example 11. Cell Uptake Assays for Compound 1

### Cell Culture

### 1.1 Thawing Method

1.1.1 15 mL of cell culture medium was placed into a T225 flask.
1.1.2 The flask was placed in a humidified 37° C., 5% CO₂ incubator for 15 minutes to allow medium to equilibrate to the proper pH and temperature.
1.1.3 The vial was removed from liquid nitrogen and thawed rapidly by placing at 37° C. in a water bath with gentle agitation for 1-2 minutes and then decontaminated by wiping with 70% ethanol before opening in a Class II biological safety cabinet.
1.1.4 The vial contents were transferred dropwise into 10 mL of cell culture medium in a sterile 15 mL conical tube.
1.1.5 The cells were centrifuged at 1,300 rpm for 5 minutes.
1.1.6 The supernatant was aspirated and re-suspended the cells and transferred the cells into T225 flask containing cell culture medium.

### 1.2 Propagation Method

1.2.1 Culture medium were renewed every 2-3 days.
1.2.2 Keep the flask in a humidified 37° C., 5% CO₂ incubator

### Assay Procedures

### 2.1 Cell Seeding

Cells were harvested and diluted in culture medium to the designated concentration. Then the cells were cultured in T25 flasks, one T25 flask for one sample.
2.2 Formulation of test compounds
2.2.1 L-Boronophenylalanine (L-BPA, 4-boron-L-phenylalanine, Purity: 95%, Chemical formula: C₉H₁₂BNO₄, MW: 209.01, CAS: 76410-58-7) and fructose were dissolved in PBS at a molar ratio of 1:5, and then a 1.27 M ratio of 1 N NaOH is added. The mixture was stirred until L-BPA is completely dissolved, and the pH value was titrated to 7.2-7.4 with 1N HCl.
2.2.2 For test compounds other than BPA, stock solutions were prepared of each compound in DMSO or fructose formulation at a concentration of 20 mM (such that final % DMSO in the cellular assay medium=0.5%)

### 2.3 Compound Treatment

2.3.1 The cells were seeded overnight (12-18 hours) to allow the cells to adhere well.
2.3.2 The stock solution of each compound was added to each T25 flask such that the final concentration for each compound is 100 uM (0.1 mM).
2.3.3 The flasks were placed in a 37° C., 5% CO₂ incubator for 1 hour, 4 hours and 24 hours.
2.3.4 After treatment for 1 hour, 4 hour, and 24 hours, the cells were collected by using 0.25% Trypsin with 0.53 mM EDTA.
   2.3.5 Centrifuged at 1,300 rpm for 5 min to collect cell pellets.

### Results

The compounds disclosed herein were selectively taken up by the representative human cancer cell lines SAS (head & neck cancer), B 16F10 (melanoma), and U87-MG (glioblastoma) relative to a representative normal human cell line (NIH-3T3). Compound 1, in addition to showing selective partitioning into cancer cells relative to normal cells, showed increased uptake in cancer cells relative to Comparator Compounds A and B.

In one experiment, cell uptake data for Compound 1 and L-BPA (Comparator Compound A) was determined separately in the same batch of cells. Table 1 and Table 2 show the cell uptake data for Compound 1 and L-BPA, respectively.

TABLE 1
Cell uptake of Compound 1

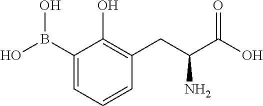 [SMILES: N[C@@H](Cc1cccc(B(O)O)c1O)C(=O)O]

0.1 mM cells concentration-time data

| | Mean (ng/10⁶ cell) | | | |
|---|---|---|---|---|
| Time (h) | SAS | B16F10 | NIH-3T3 | U87-MG |
| 1.00 | 863 | 158 | 109 | 175 |
| 4.00 | 1400 | 282 | 194 | 713 |
| 24.0 | 2625 | 669 | 122 | 1315 |

TABLE 2
Cell uptake of Comparator Compound A (L-BPA)

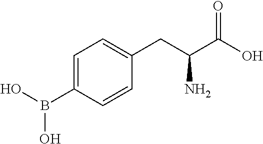 [SMILES: N[C@@H](Cc1ccc(B(O)O)cc1)C(=O)O]

L-Boronophenylalanine 0.1 mM cells concentration-time data

| | Mean (ng/10⁶ cell) | | | |
|---|---|---|---|---|
| Time (h) | SAS | B16F10 | NIH-3T3 | U87-MG |
| 1.00 | 425 | 292 | 126 | 391 |
| 4.00 | 379 | 247 | 127 | 471 |
| 24.0 | 423 | 456 | 111 | 546 |

In another experiment, cell uptake data for Comparator Compound B and L-BPA (Comparator Compound A) was determined separately in the same batch of cells. Table 3 and Table 4 show the cell uptake data for Compound B and L-BPA, respectively.

TABLE 3
Cell uptake of Comparator Compound B;
(compound disclosed in WO 2020/180390
incorporated by reference)

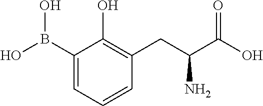 [SMILES: N[C@@H](Cc1cccc(B(O)O)c1O)C(=O)O]

0.1 mM cells concentration-time data

| | Mean (ng/10⁶ cell) | | | |
|---|---|---|---|---|
| Time (h) | SAS | B16F10 | NIH-3T3 | U87-MG |
| 1.00 | 510 | 131 | 186 | 380 |
| 4.00 | 487 | 248 | 122 | 519 |
| 24.0 | 1465 | 662 | 217 | 250 |

TABLE 4
Cell uptake of Comparator Compound A (L-BPA)
L-Boronophenylalanin 0.1 mM cells concentration-time data

| | |
|---|---|
| Time | Mean (ng/10⁶ cell) |

| (h) | SAS | B16F10 | NIH-3T3 | U87-MG |
|---|---|---|---|---|
| 1.00 | 425 | 187 | 215 | 343 |
| 4.00 | 352 | 224 | 287 | 440 |
| 24.0 | 444 | 467 | 176 | 204 |

### Example 12. Further Cell Uptake Assays

In further experiments, cell uptake data for Compounds 3,4, 6, and 8 and Comparator Compound A was obtained (see Tables 5-8).

TABLE 5
Cell uptake of Compound 3 and Comparator Compound A

| Compound 3 0.1 mM cells concentration-time data | | | | | L-BPA 0.1 mM cells concentration-time data | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1 | 1320 | 66.5 | 105 | 307 | 1 | 407 | 840 | 228 | 233 |
| 4 | 1295 | 255 | 196 | 621 | 2 | 394 | 986 | 177 | 223 |
| 24 | 1580 | 196 | 182 | 555 | 24 | 501 | 790 | 145 | 195 |

TABLE 6
Cell uptake of Compound 4 and Comparator Compound A

| Compound 4 0.1 mM cells concentration-time data | | | | | L-BPA 0.1 mM cells concentration-time data | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1 | 33.1 | 29.5 | 28.3 | 14.3 | 1 | 260 | 112 | 54.8 | 233 |
| 4 | 191 | 39.1 | 36.9 | 40 | 4 | 251 | 188 | 77.5 | 260 |
| 24 | 886 | 48.1 | 55.3 | 117 | 24 | 261 | 222 | 136 | 213 |

TABLE 7
Cell uptake of Compound 6 and Comparator Compound A

| Compound 6 0.1 mM cells concentration-time data | | | | | L-BPA 0.1 mM cells concentration-time data | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1 | 373 | 39.6 | 20.3 | 58.6 | 1 | 254 | 146 | 134 | 196 |
| 4 | 796 | 75.9 | 59.8 | 171 | 4 | 219 | 191 | 160 | 123 |
| 24 | 1735 | 222 | 50.8 | 244 | 24 | 345 | 302 | 185 | 181 |

TABLE 8
Cell uptake of Compound 8 and Comparator Compound A

| Compound 8 0.1 mM cells concentration-time data | | | | | L-BPA 0.1 mM cells concentration-time data | | | | |
|---|---|---|---|---|---|---|---|---|---|
| Time | Mean (ng/10⁶ cell) | | | | Time | Mean (ng/10⁶ cell) | | | |
| (h) | SAS | B16F10 | NIH-3T3 | U87-MG | (h) | SAS | B16F10 | NIH-3T3 | U87-MG |
| 1 | 561 | 115 | 51.7 | 173 | 1 | 218 | 135 | 183 | 297 |
| 4 | 693 | 185 | 119 | 505 | 4 | 217 | 185 | 162 | 397 |
| 24 | 1605 | 615 | 101 | 545 | 24 | 336 | 339 | 263 | 334 |

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (I): 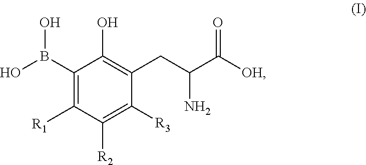
wherein
R₁, R₂, and R₃ are each independently selected from —H, halo, alkyl, haloalkyl, and cycloalkyl; and
the compound is racemic, enriched in one enantiomer, or a single enantiomer;
or a pharmaceutically acceptable salt thereof.

2. The compound of claim 1 having the structure of Formula (IA): 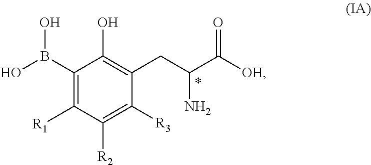
wherein * indicates a chiral carbon with an absolute configuration of (S) or (R); and the compound is not racemic.

3. The compound of claim 2, wherein the absolute configuration of the chiral carbon is (S).

4. The compound of claim 1 having the structure of Formula (IB):

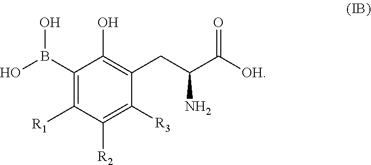

5. The compound of claim 2, wherein the absolute configuration of the chiral carbon is (R).

6. The compound of claim 1 having the structure of Formula (IC):

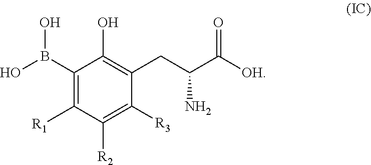

7. The compound of claim 1, wherein R₁, R₂, and R₃ are each independently selected from —H, —Cl, —F, —CF₃, and —CH₃.

8. The compound of claim 1, wherein at least one R₁, R₂, and R₃ is not —H.

9. The compound of claim 1, wherein one and only one of R₁, R₂, and R₃ is not —H.

10. The compound of claim 1 having the structure: 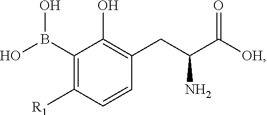
provided R₁ is not —H; or 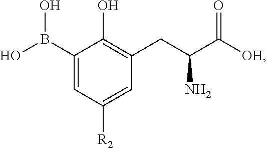
provided R₂ is not —H; or 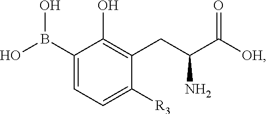
provided R₃ is not —H.

11. (canceled)

12. (canceled)

13. The compound of claim 1 having the structure selected from: 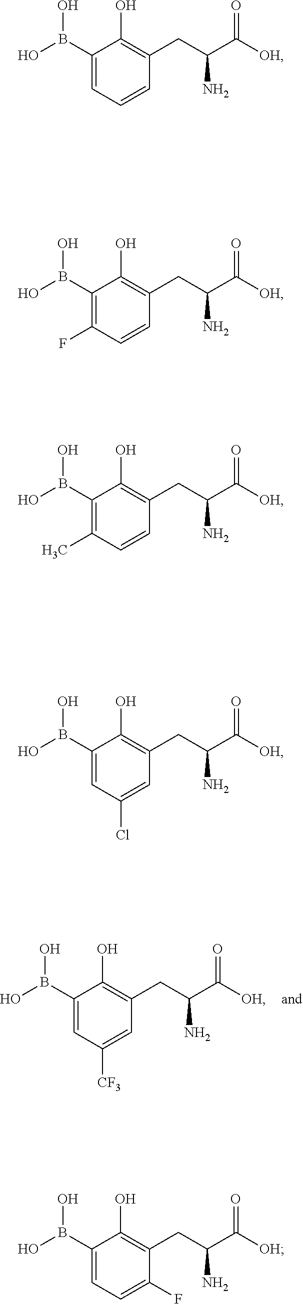
or a pharmaceutically acceptable salt thereof.

14. (canceled)

15. The compound of claim 1 having the structure selected from: 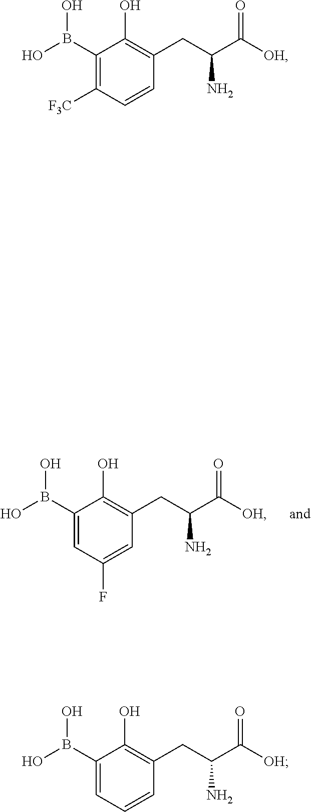 [SMILES: N[C@@H](Cc1ccc(C(F)(F)F)c(B(O)O)c1O)C(=O)O ; N[C@@H](Cc1cc(F)cc(B(O)O)c1O)C(=O)O ; "and" ; N[C@H](Cc1cccc(B(O)O)c1O)C(=O)O]
or a pharmaceutically acceptable salt thereof.

16. The compound of claim 1, wherein the boron atom in the compound is ¹⁰B.

17. A pharmaceutical composition, comprising a compound claim 1; and a pharmaceutical acceptable excipient.

18. The pharmaceutical composition of claim 17, further comprising a saccharide, a polyhydroxy acid, or a sugar alcohol.

19. (canceled)

20. (canceled)

21. A method of treating cancer, comprising:
i) administering to a subject in need thereof an effective amount of a compound of claim 1, wherein the compound accumulates in a plurality of cancer cells in the subject; and
ii) irradiating the plurality of cancer cells with neutrons.

22. (canceled). 23. (Canceled)

24. The method of claim 1, wherein the compound or the composition is administered intravenously.

25. (canceled)

26. The method of claim 1, wherein in step (i) the compound is administered at about 100 mg/kg/h to about 500 mg/kg/h for a first period of time.

27. (canceled)

28. (canceled)

29. The method of claim 1, wherein in step (ii) the compound is administered at about 50 mg/kg/h to about 150 mg/kg/h or about 100 mg/kg/h to about 200 mg/kg/h for a second period of time.

30. (canceled)

31. (canceled)

32. (canceled)

33. The method of claim 21, wherein the cancer is selected from head and neck cancer, glioblastoma, melanoma, sarcoma, breast cancer, meningioma, lung cancer, mesothelioma, hepatocellular carcinoma, and extramammary Paget disease.

34. (canceled)
